(19) United States
(12) Patent Application Publication
(10) Pub. No.: US 2025/0011727 A1
(43) Pub. Date: Jan. 9, 2025

(54) HYDROGEL STRUCTURES AND METHODS OF MAKING AND USE THEREOF

(71) Applicant: GEORGIA TECH RESEARCH CORPORATION, Atlanta, GA (US)

(72) Inventors: Andres J. GARCIA, Atlanta, GA (US); Ankur SINGH, Marietta, GA (US); Adriana MULERO-RUSSE, Atlanta, GA (US); Ana MORA BOZA, Atlanta, GA (US); Maria M. CORONEL, Atlanta, GA (US); Valeria M. JUAREZ, Atlanta, GA (US)

(21) Appl. No.: 18/705,735

(22) PCT Filed: Oct. 28, 2022
(86) PCT No.: PCT/US2022/048199

§ 371 Date: Apr. 29, 2024

Related U.S. Application Data
(60) Provisional application No. 63/273,222, filed on Oct. 29, 2021.
(60) Provisional application No. 63/273,224, filed on Oct. 29, 2021.

(51) Int. Cl. C12N 5/071; C12M 1/00; C12M 1/12; C12M 3/00

(52) U.S. Cl. CPC C12N 5/0697; C12M 21/08; C12M 25/04; C12M 29/10; C12N 2513/00; C12N 2533/30; C12N 2533/50

(57) ABSTRACT

Disclosed herein are hydrogel structures and methods of making and use thereof. For example, disclosed herein are methods of making a device comprising a hydrogel matrix and a first chamber in the hydrogel matrix, the hydrogel matrix being derived from a prepolymer and the first chamber being perfusable. The methods can, for example, comprise blocking a first portion of a pre-polymerization solution with a first photomask, the pi-polymerization solution comprising the prepolymer, such that the pre-polymerization solution comprises an exposed portion and a first blocked portion. The methods can further comprise irradiating the exposed portion of the pi-polymerization solution and the first photomask with electromagnetic radiation, the first photomask being substantially opaque to the electromagnetic radiation. In some examples, the prepolymer within the exposed portion of pre-polymerization solution photopolymerizes to form the hydrogel matrix and the prepolymer within the first blocked portion does not photopolymerize and forms the first chamber.

### CROSS-REFERENCE TO RELATED APPLICATIONS

This application claims the benefit of priority to U.S. Provisional Application No. 63/273,222 filed Oct. 29, 2021 and U.S. Provisional Application 63/273,224 filed Oct. 29, 2021, both of which are hereby incorporated herein by reference in their entireties.

### BACKGROUND

Fabrication of current perfusable organ-on-a-chip platforms based on hydrogels: involve painstaking, time-consuming, and laser-based equipment-intensive methodologies; and are limited to natural, biological matrices. Faster and easier methods based on synthetic hydrogels as well as strategies to pattern cells within organ-on-a-chip devices are needed. The compositions, devices, and methods disclosed herein address these and other needs.

### SUMMARY

In accordance with the purposes of the disclosed compositions, devices, and methods as embodied and broadly described herein, the disclosed subject matter relates to hydrogel structures and methods of making and use thereof.

For example, disclosed herein are methods of making a device comprising a hydrogel matrix and a first chamber in the hydrogel matrix, the hydrogel matrix being derived from a prepolymer and the first chamber being perfusable. The methods can, for example, comprise blocking a first portion of a pre-polymerization solution with a first photomask, the pre-polymerization solution comprising the prepolymer, such that the pre-polymerization solution comprises an exposed portion and a first blocked portion. The methods can further comprise irradiating the exposed portion of the pre-polymerization solution and the first photomask with electromagnetic radiation, the first photomask being substantially opaque to the electromagnetic radiation. In some examples, the prepolymer within the exposed portion of pre-polymerization solution photopolymerizes to form the hydrogel matrix and the prepolymer within the first blocked portion does not photopolymerize and forms the first chamber.

Also disclosed herein are methods of making a device comprising a hydrogel matrix derived from a prepolymer; a first chamber in the hydrogel matrix, the first chamber being perfusable; and a second chamber in the hydrogel matrix, the second chamber being perfusable and fluidly independent from the first chamber. The methods can, for example, comprise blocking a first portion of a pre-polymerization solution with a first photomask and a second portion of the pre-polymerization solution with a second photomask, the pre-polymerization solution comprising the prepolymer, such that the pre-polymerization solution comprises an exposed portion, a first blocked portion, and a second blocked portion. The methods can further comprise irradiating the exposed portion of the pre-polymerization solution, the first photomask, and the second photomask with electromagnetic radiation, the first photomask and the second photomask being substantially opaque to the electromagnetic radiation. In some examples, the prepolymer within the exposed portion of pre-polymerization solution photopolymerizes to form the hydrogel matrix, the prepolymer within the first blocked portion does not photopolymerize and forms the first chamber, and the prepolymer within the second blocked portion does not photopolymerize and forms the second chamber.

In some examples, the methods can further comprise, after irradiation, removing the first photomask and the second photomask (when present).

In some examples, the methods can further comprise, after irradiation, rinsing the hydrogel device to remove any remaining pre-polymerization solution and/or prepolymer.

In some examples, the methods can further comprise disposing the pre-polymerization solution in a mold defining a shape before blocking the first portion of the pre-polymerization solution with the first photomask.

In some examples, the hydrogel matrix comprises a synthetic hydrogel.

In some examples, the hydrogel matrix is derived from a prepolymer having a molecular weight of from 0.5 to 200 kilodaltons (kDa). In some examples, the hydrogel matrix is derived from a prepolymer having a molecular weight of from 2 kDa to 40 kDa, from 2 kDa to 25 kDa, or from 2 kDa to 10 kDa.

In some examples, the hydrogel matrix is derived from a branched prepolymer. In some examples, the prepolymer has 3 or more branches, 4 or more branches, or 8 or more branches.

In some examples, the hydrogel matrix is derived from a prepolymer comprising polyethylene glycol or a derivative thereof.

In some examples, the hydrogel matrix is derived from a prepolymer comprising poly(ethylene glycol) acrylate, poly(ethylene glycol) diacrylate (PEGDA), poly(ethylene glycol) dimethacrylate (PEGDMA), poly(ethylene glycol) diacrylamide (PEGDAAm), polyethylene glycol norbornene, polyethylene glycol dithiol. PEG based peptide conjugates, cell-adhesive poly(ethylene glycol), MMP-sensitive poly(ethylene glycol), PEGylated fibrinogen, PEGylated collagen, PEGylated laminin, or a combination thereof. In some examples, the hydrogel matrix is derived from a prepolymer comprising poly(ethylene glycol) vinyl sulfone, poly(ethylene glycol) acrylate, poly(ethylene glycol) maleimide, poly(ethylene glycol) norbornene, or a combination thereof. In some examples, the hydrogel matrix is derived from a prepolymer comprising poly(ethylene glycol) maleimide, poly(ethylene glycol) norbornene, or a combination thereof. In some examples, the hydrogel matrix is derived from a prepolymer comprising poly(ethylene glycol) norbornene.

In some examples, the pre-polymerization solution comprises the prepolymer in an amount of from 1 wt. % to 30 wt. %. In some examples, the pre-polymerization solution comprises the prepolymer in an amount of from 4 wt. % to 10 wt. % or from 1 wt. % to 5 wt. %.

In some examples, the pre-polymerization solution further comprises a crosslinker and the hydrogel is further derived from the crosslinker. In some examples, the crosslinker is a multifunctional crosslinker. In some examples, the crosslinker comprises a multifunctional thiol. In some examples, the crosslinker comprises a dithiol.

In some examples, the hydrogel matrix is further derived from one or more additional components, such as one or more additional monomers, prepolymers, ligands, chemical agents, therapeutic agents, photoinitiators, or a combination thereof. In some examples, the pre-polymerization solution further comprises said one or more additional components.

In some examples, the hydrogel matrix is further derived from a natural or biological prepolymer. In some examples, the natural or biological prepolymer comprises fibrinogen, collagen, or a combination thereof.

In some examples, the pre-polymerization solution further comprises a photoinitiator.

In some examples, the hydrogel matrix further comprises a chemical agent, a therapeutic agent, or a combination thereof dispersed therein. In some examples, the chemical agent and/or the therapeutic agent is/are dispersed inhomogeneously within the hydrogel matrix. In some examples, the chemical agent and/or therapeutic agent have a concentration that varies across the hydrogel matrix, such that the chemical agent and/or the therapeutic agent has a compositional gradient across the hydrogel matrix. In some examples, the pre-polymerization solution further comprises the chemical agent and/or the therapeutic agent.

In some examples, the electromagnetic radiation comprises UV radiation.

In some examples, the electromagnetic radiation comprises one or more wavelengths of from 10 nm to 900 nm. In some examples, the electromagnetic radiation comprises one or more wavelengths of from 100 nm to 900 nm or from 100 nm to 400 nm.

In some examples, the electromagnetic radiation is provided by a light source and the light source is an artificial light source. In some examples, the light source comprises a light emitting diode (LED), a lamp, a laser, or a combination thereof.

In some examples, the exposed portion of the pre-polymerization solution photopolymerizes in an amount of time of from 1 millisecond to 1 hour. In some examples, the exposed portion of the pre-polymerization solution photopolymerizes in an amount of time of from 1 millisecond to 1 minute, from 1 millisecond to 10 seconds, or from 1 millisecond to 1 second.

In some examples, the hydrogel matrix exhibits a swelling of 10%/0 or less.

In some examples, the hydrogel matrix exhibits a shape fidelity of 50% or more, 75% or more, or 80% or more.

In some examples, the hydrogel matrix has a storage modulus of from greater than 0 Pa to 5000 Pa. In some examples, the hydrogel matrix has a storage modulus of from greater than 0 Pa to 600 Pa or from greater than 0 Pa to 300 Pa.

In some examples, the hydrogel matrix is configured to be stable for an amount of time of from 1 day to 3 months. In some examples, the hydrogel matrix is configured to be stable for an amount of time of 1 to 7 days.

In some examples, the hydrogel matrix is continuous.

In some examples, the hydrogel matrix is monolithic.

In some examples, the hydrogel matrix is porous.

In some examples, the hydrogel matrix is biocompatible.

In some examples, the hydrogel matrix is biodegradable.

In some examples, the hydrogel matrix comprises a photopolymerized polymer network.

In some examples, the hydrogel matrix comprises a cross-linked polymer network.

Also disclosed herein are devices made by any of the methods disclosed herein. In some examples, the device is a microfluidic device.

Also disclosed herein are methods of use of any of the devices disclosed herein.

In some examples, the methods comprise using the device for diagnostics, disease modeling, regenerative medicine, drug screening, tissue modeling, or a combination thereof.

In some examples, the methods comprise using the device as a biomaterial substrate or scaffold, a cell culture substrate or platform, or a combination thereof.

In some examples, the method comprises seeding the device (e.g., the first and/or second chamber) with a cell and/or biomaterial, and perfusing the device (e.g., the first and/or second chamber) with a solution. In some examples, the solution comprises cell culture media.

In some examples, the method comprises using the device as a cell culture substrate for immunocytes, B cells, lymph cells, dendritic cells, lung cells, intestinal cells, endothelial cells, hepatocytes, kidney epithelial cells, or a combination thereof.

In some examples, the cultured cells exhibit a cell viability of 50% or more, 65% or more, or 80% or more after 4 days or more in the device.

In some examples, the method comprises using the device to grow an organoid.

In some examples, the method comprises using the device to grow three-dimensional cell clusters.

In some examples, the method comprises using the device to grow a human organoid.

In some examples, the method comprises using the device to grow a lymphoid follicle organoid, a tonsil organoid, an intestinal organoid, a lung organoid, or a combination thereof.

In some examples, the method comprises using the device to grow a human intestinal organoid.

In some examples, the method comprises using the device as a cell culture substrate and the cultured cells exhibit cell phenotype differentiation.

In some examples, the method comprises using the device as a cell culture substrate and the cultured cells comprise viable intestinal cells displaying appropriate apical and basolateral marker localization, differentiated epithelial cells, or a combination thereof.

In some examples, the method comprises using the device as a cell culture substrate and the cultured cells colonize the device tri-dimensionally.

Also disclosed herein are articles of manufacture comprising any of the devices disclosed herein. In some examples, the article comprises a biomaterial substrate or scaffold, a cell culture substrate or platform, or a combination thereof.

Additional advantages of the disclosed compositions, devices, and methods will be set forth in part in the description which follows, and in part will be obvious from the description.

The advantages of the disclosed compositions, devices, and methods will be realized and attained by means of the elements and combinations particularly pointed out in the appended claims. It is to be understood that both the foregoing general description and the following detailed description are exemplary and explanatory only and are not restrictive of the disclosed compositions, devices, and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1. Schematic diagram of engineering a multiorgan platform.

FIG. 2. Schematic diagram of an immune follicle-on-a-chip.

FIG. 3. Schematic diagram of lymphoid tissue integrated gut organoids.

FIG. 4. Schematic diagram of approach towards biomaterial-based human lymphoid follicle organoids.

FIG. 5. Schematic diagram of approach towards biomaterial-based human lymphoid follicle organoids.

FIG. 6. Germinal center B cell count.

FIG. 7. Schematic diagram of approach towards biomaterial-based human lymphoid follicle organoids.

FIG. 8. EMC-dependent/independent spreading of stromal CD40L cells and FDCs.

FIG. 9. EMC-dependent/independent survival of stromal CD40L cells and FDCs FIG. 10. Temporal stability and impact of polymer weight % on hydrogel stiffness.

FIG. 11. Images of primary human B cell survival over time.

FIG. 12. Comparison of primary human B cell survival for tonsils and PBMCs.

FIG. 13. Primary human B cell survival for tonsils.

FIG. 14. Primary human B cell survival for PBMCs.

FIG. 15. Schematic diagram of microfluidic devices for lymphoid follicle-on-a-chip engineering.

FIG. 16. Images and shape fidelity of various microfluidic devices for lymphoid follicle-on-a-chip engineering.

FIG. 17. Schematic diagram of synthesis of thiol-norbornene photoclickable PEG hydrogels.

FIG. 18. Cell survival in various hydrogels.

FIG. 19. Degradation of hydrogel over time.

FIG. 20. Cell survival in various hydrogels.

FIG. 21. Schematic diagram of developing lymphoid tissue-integrated gut organoids to study vaccine mediated immunity.

FIG. 22. PEG-4MAL density and adhesive peptide control.

FIG. 23. HIO viability and development validation for hiPSC in PEG-4MAL.

FIG. 24. HIO viability and development validation for hiPSC in PEG-4MAL.

FIG. 25. Schematic diagram of investigating effect of PEG-4MAL density on H10 generation.

FIG. 26. Images of HIOs in different hydrogels.

FIG. 27. Images of intestinal cells from hiPSCs grown in hydrogels.

FIG. 28. Images of gut organoid-on-a-chip microchip system comprising an elastomeric device with a central hydrogel chamber for subsequent organoid culture and perfusion.

FIG. 29. Characterization of composite PEG-4aNB hydrogel bulk and PEG-4MAL microgels containing human B cells.

FIG. 30. Schematic diagram of HIOs and uses thereof.

FIG. 31. Schematic diagram of the synthetic hydrogel photopolymerization mechanism.

FIG. 32. Schematic diagram of photopatterning PEG-4NB hydrogels for the fabrication of perfusable mini-gut structures using UV-light and a photomask.

FIG. 33. Effect of crosslinker and wt. % on hydrogel swelling.

FIG. 34. Effect of PEG-4NB molecular weight on hydrogel swelling.

FIG. 35. Effect of temperature on hydrogel swelling.

FIG. 36. Shape fidelity of hydrogels.

FIG. 37. Image of example device with complex geometry.

FIG. 38. Schematic diagram of seeding HIOs in a gut-on-a-chip device.

FIG. 39. Images illustrating importance of media perfusion on cell survival.

FIG. 40. Images illustrating importance of media perfusion on cell survival and device coverage.

FIG. 41. Image showing growth of HIO colonization of hydrogel device.

FIG. 42. Images of hydrogel device with bullseye design.

FIG. 43. Image of hydrogel device with bullseye design.

FIG. 44. Image of hydrogel device with bullseye design.

FIG. 45. Fluorescence over time of hydrogel device with bullseye design.

FIG. 46. Schematic diagram of tangential flow in hydrogel device with bullseye design.

FIG. 47. Schematic diagram of orthogonal flow in hydrogel device with bullseye design.

FIG. 48. Images illustrating tangential flow in hydrogel device with bullseye design.

FIG. 49. Images illustrating orthogonal flow in hydrogel device with bullseye design

FIG. 50. MFI for hydrogel device with bullseye design using tangential or orthogonal flow.

FIG. 51. B cell viability in hydrogel devices of different compositions.

FIG. 52. GCBs in hydrogel devices of different compositions.

FIG. 53. Median fluorescence intensity in hydrogel devices of different compositions.

FIG. 54. Cell proliferation in hydrogel devices of different compositions.

FIG. 55. Images of cell proliferation and clustering overtime in PEG-4MAL based hydrogel device.

FIG. 56. Images of cell proliferation and clustering overtime in PEG-4NB based hydrogel device with PEG-4NB:Fibrin/collagen ratio of 1:0.

FIG. 57. Images of cell proliferation and clustering overtime in PEG-4NB based hydrogel device with PEG-4NB:Fibrin/collagen ratio of 3:1.

FIG. 58. Images of cell proliferation and clustering overtime in PEG-4NB based hydrogel device with PEG-4NB:Fibrin/collagen ratio of 1:1.

FIG. 59. B cell viability in PEG-4NB hydrogel devices with varying amount of FC.

FIG. 60. B cell viability in PEG-4NB hydrogel devices with varying amount of FC.

FIG. 61. B cell viability in PEG-4NB hydrogel devices with varying amount of FC.

FIG. 62. B cell viability in PEG-4NB hydrogel devices with varying amount of FC.

FIG. 63. Schematic diagram of an example hydrogel device.

FIG. 64. Image showing Human B and T cell migration induced by CXCL12 gradient within hydrogel device.

FIG. 65. Schematic diagram of an example hydrogel device.

FIG. 66. Images illustrating cell differentiation in hydrogel devices comprising PEG-4NB, fibrinogen, and collagen relative to one comprising PEG-4NB.

FIG. 67. Schematic diagram of patterning of natural-based matrices using PALM microbeam Zeiss microscope as a dissection tool.

FIG. 68. Immunostaining gut on a chip after 4 days in culture.

FIG. 69. Immunostaining gut on a chip after 4 days in culture.

FIG. 70. Immunostaining gut on a chip after 4 days in culture.

FIG. 71. Images illustrating colonization of hydrogel device.

FIG. 72. Images illustrating colonization of hydrogel device.

FIG. 73. Image illustrating colonization of hydrogel device.

FIG. 74. Image illustrating colonization of hydrogel device.

FIG. 75. Image illustrating colonization of hydrogel device.

FIG. 76. Image illustrating colonization of hydrogel device at a first time point.

FIG. 77. Image illustrating colonization of hydrogel device at a second time point, which is a later time point relative to FIG. 76.

FIG. 78. Image illustrating colonization of hydrogel device at a third time point, which is a later time point relative to FIG. 77.

FIG. 79. Image illustrating colonization of hydrogel device at a fourth time point, which is a later time point relative to FIG. 78.

FIG. 80. Image illustrating colonization of hydrogel device at a fifth time point, which is a later time point relative to FIG. 79.

FIG. 81. Image illustrating colonization of hydrogel device at a sixth time point, which is a later time point relative to FIG. 80.

FIG. 82. Image illustrating colonization of hydrogel device at a seventh time point, which is a later time point relative to FIG. 81.

FIG. 83. Image illustrating colonization of hydrogel device at an eighth time point, which is a later time point relative to FIG. 82.

FIG. 84. Immunostaining of hiPSCs-derived spheroids in tubular gut on a chip device after 7 days of culture.

FIG. 85. Immunostaining of hiPSCs-derived spheroids in tubular gut on a chip device after 7 days of culture.

### DETAILED DESCRIPTION

The compositions, devices, and methods described herein may be understood more readily by reference to the following detailed description of specific aspects of the disclosed subject matter and the Examples included therein.

Before the present compositions, devices, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Exemplary” means “an example of” and is not intended to convey an indication of a preferred or ideal embodiment. “Such as” is not used in a restrictive sense, but for explanatory purposes.

Values can be expressed herein as an “average” value. “Average” generally refers to the statistical mean value.

By “substantially” is meant within 5%, e.g., within 4%, 3%, 2%, or 1%.

“Exemplary” means “an example of” and is not intended to convey an indication of a preferred or ideal embodiment. “Such as” is not used in a restrictive sense, but for explanatory purposes.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

“Biocompatible” and “biologically compatible”, as used herein, generally refer to compounds and/or compositions that are, along with any metabolites or degradation products thereof, generally non-toxic to normal cells and tissues, and which do not cause any significant adverse effects to normal cells and tissues when cells and tissues are incubated (e.g., cultured) in their presence.

The term “biodegradable” as used herein refers to a material or substance wherein physical dissolution and/or chemical degradation is effected under physiological conditions.

As used herein, “antimicrobial” refers to the ability to treat or control (e.g., reduce, prevent, treat, or eliminate) the growth of a microbe at any concentration. Similarly, the terms “antibacterial,” “antifungal,” and “antiviral” refer to the ability to treat or control the growth of bacteria, fungi, and viruses at any concentration, respectively.

As used herein, “reduce” or other forms of the word, such as “reducing” or “reduction,” refers to lowering of an event or characteristic (e.g., microbe population/infection). It is understood that the reduction is typically in relation to some standard or expected value. For example, “reducing microbial infection” means reducing the spread of a microbial infection relative to a standard or a control.

As used herein, “prevent” or other forms of the word, such as “preventing” or “prevention,” refers to stopping a particular event or characteristic, stabilizing or delaying the development or progression of a particular event or characteristic, or minimizing the chances that a particular event or characteristic will occur. “Prevent” does not require comparison to a control as it is typically more absolute than, for example, “reduce.” As used herein, something could be reduced but not prevented, but something that is reduced could also be prevented. Likewise, something could be prevented but not reduced, but something that is prevented could also be reduced.

As used herein, “treat” or other forms of the word, such as “treated” or “treatment,” refers to administration of a composition or performing a method in order to reduce, prevent, inhibit, or eliminate a particular characteristic or event (e.g., microbe growth or survival). The term “control” is used synonymously with the term “treat.”

The term “anticancer” refers to the ability to treat or control cellular proliferation and/or tumor growth at any concentration.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

### Chemical Definitions

Unless otherwise defined, all technical and scientific terms used herein have the same meaning as commonly understood by one of ordinary skill in the art to which this invention belongs.

The organic moieties mentioned when defining variable positions within the general formulae described herein (e.g., the term “halogen”) are collective terms for the individual substituents encompassed by the organic moiety. The prefix Cₙ-Cₘ preceding a group or moiety indicates, in each case, the possible number of carbon atoms in the group or moiety that follows.

The term “ion,” as used herein, refers to any molecule, portion of a molecule, cluster of molecules, molecular complex, moiety, or atom that contains a charge (positive, negative, or both at the same time within one molecule, cluster of molecules, molecular complex, or moiety (e.g., zwitterions)) or that can be made to contain a charge. Methods for producing a charge in a molecule, portion of a molecule, cluster of molecules, molecular complex, moiety, or atom are disclosed herein and can be accomplished by methods known in the art, e.g., protonation, deprotonation, oxidation, reduction, alkylation, acetylation, esterification, de-esterification, hydrolysis, etc.

The term “anion” is a type of ion and is included within the meaning of the term “ion.” An “anion” is any molecule, portion of a molecule (e.g., zwitterion), cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

The term “cation” is a type of ion and is included within the meaning of the term “ion.” A “cation” is any molecule, portion of a molecule (e.g., zwitterion), cluster of molecules, molecular complex, moiety, or atom, that contains a net positive charge or that can be made to contain a net positive charge. The term “cation precursor” is used herein to specifically refer to a molecule that can be converted to a cation via a chemical reaction (e.g., protonation or alkylation).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, heptyl, octyl, nonyl, decyl, dodecyl, tetradecyl, hexadecyl, eicosyl, tetracosyl, and the like. Alkyl substituents may be unsubstituted or substituted with one or more chemical moieties. The alkyl group can be substituted with one or more groups including, but not limited to, hydroxyl, halogen, acyl, alkyl, alkoxy, alkenyl, alkynyl, aryl, heteroaryl, aldehyde, amino, cyano, carboxylic acid, ester, ether, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, or C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure —CH═CH—CH₃; and 2-propenyl refers to a group with the structure —CH₂—CH═CH₂. Asymmetric structures such as (Z¹Z²)C═C(Z³Z⁴) are intended to include both the E and 2 isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₄, C₂-C₂₀, C₂-C₁₈, C₂-C₁₀, C₂-C₁₄, C₂-C₁₂, C₂-C₁₀, C₂-C₈, C₂-C₆, or C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol, as described below.

As used herein, the term “aryl,” as well as derivative terms such as aryloxy, refers to groups that include a monovalent aromatic carbocyclic group of from 3 to 50 carbon atoms. Aryl groups can include a single ring or multiple condensed rings. In some embodiments, aryl groups include C₆-C₁₀ aryl groups. Examples of aryl groups include, but are not limited to, benzene, phenyl, biphenyl, naphthyl, tetrahydronaphthyl, phenylcyclopropyl, phenoxybenzene, and indanyl. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is also included in the term “aryl,” defines a group that contains an aromatic group that does not contain a heteroatom. The aryl substituents may be unsubstituted or substituted with one or more chemical moieties. Examples of suitable substituents include, for example, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems (e.g., monocyclic, bicyclic, tricyclic, polycyclic, etc.) that can be substituted or unsubstituted. A cyclic group can contain one or more aryl groups, one or more non-aryl groups, or one or more aryl groups and one or more non-aryl groups.

The term “acyl” as used herein is represented by the formula —C(O)Z¹ where Z¹ can be a hydrogen, hydroxyl, alkoxy, alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a shorthand notation for C═O.

The term “acetal” as used herein is represented by the formula (Z¹Z²)C═OZ³)(═OZ⁴), where Z¹, Z², Z³, and Z⁴ can be, independently, a hydrogen, halogen, hydroxyl, alkyl, alkenyl, alkynyl, aryl, heteroaryl, cycloalkyl, cycloalkenyl, heterocycloalkyl, or heterocycloalkenyl group described above.

The term “alkanol” as used herein is represented by the formula Z¹OH, where Z¹ can be an alkyl, alkenyl, alkynyl, aryl, heteroaryl, cycloalkyl, cycloalkenyl, heterocycloalkyl, or heterocycloalkenyl group described above.

As used herein, the term “alkoxy” as used herein is an alkyl group bound through a single, terminal ether linkage; that is, an “alkoxy” group can be defined as to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H. Throughout this specification “C(O)” is a shorthand notation for C═O.

The terms “amine” or “amino” as used herein are represented by the formula —NZ¹Z²Z³, where Z¹, Z², and Z³ can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The terms “amide” or “amido” as used herein are represented by the formula —C(O)NZ¹Z², where Z¹ and Z² can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The term “anhydride” as used herein is represented by the formula Z¹C(O)OC(O)Z² where Z¹ and Z², independently, can be an alkyl, alkenyl, alkynyl, aryl, heteroaryl, cycloalkyl, cycloalkenyl, heterocycloalkyl, or heterocycloalkenyl group described above.

The term “cyclic anhydride” as used herein is represented by the formula:

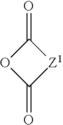

where Z¹ can be an alkyl, alkenyl, alkynyl, aryl, heteroaryl, cycloalkyl, cycloalkenyl, heterocycloalkyl, or heterocycloalkenyl group described above.

The term “azide” as used herein is represented by the formula —N═N═N.

The term “carboxylic acid” as used herein is represented by the formula —C(O)OH.

A “carboxylate” or “carboxyl” group as used herein is represented by the formula —C(O)O⁻·

A “carbonate ester” group as used herein is represented by the formula Z¹OC(O)OZ².

The term “cyano” as used herein is represented by the formula —CN.

The term “ester” as used herein is represented by the formula —OC(O)Z¹ or —C(O)Z¹, where Z¹ can be an alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “epoxy” or “epoxide” as used herein refers to a cyclic ether with a three atom ring and can represented by the formula:

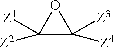

where Z¹, Z², Z³, and Z⁴ can be, independently, an alkyl, alkenyl, alkynyl, aryl, heteroaryl, cycloalkyl, cycloalkenyl, heterocycloalkyl, or heterocycloalkenyl group described above

The term “ketone” as used herein is represented by the formula Z¹C(O)Z², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “phosphonyl” is used herein to refer to the phospho-oxo group represented by the formula —P(O)(OZ¹)₂, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “silyl” as used herein is represented by the formula —SiZ¹Z²Z³, where Z¹, Z², and Z³ can be, independently, hydrogen, alkyl, alkoxy, alkenyl, alkynyl, aryl, heteroaryl, cycloalkyl, cycloalkenyl, heterocycloalkyl, or heterocycloalkenyl group described above.

The term “sulfonyl” or “sulfone” is used herein to refer to the sulfo-oxo group represented by the formula —S(O)₂Z¹, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “sulfide” as used herein is comprises the formula —S—.

The term “thiol” as used herein is represented by the formula —SH.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

“Phase,” as used herein, generally refers to a region of a material having a substantially uniform composition which is a distinct and physically separate portion of a heterogeneous system. The term “phase” does not imply that the material making up a phase is a chemically pure substance, but merely that the chemical and/or physical properties of the material making up the phase are essentially uniform throughout the material, and that these chemical and/or physical properties differ significantly from the chemical and/or physical properties of another phase within the material. Examples of physical properties include density, thickness, aspect ratio, specific surface area, porosity, and dimensionality. Examples of chemical properties include chemical composition.

The term “olefinically unsaturated group” or “ethylenically unsaturated group” is employed herein in a broad sense and is intended to encompass any groups containing a carbon-carbon double bonded group (>C═C<group). Exemplary ethylenically unsaturated groups include, but are not limited to, (meth)acrylate, (meth)acrylamide, (meth)acryloyl, allyl, vinyl, styrenyl, or other >C═C<containing groups.

“Polymer” means a material formed by polymerizing one or more monomers.

The term “(co)polymer” includes homopolymers, copolymers, or mixtures thereof.

The term “(meth)acryl . . . ” includes “acryl . . . ,” “methacryl . . . ,” or mixtures thereof.

“Molecular weight” of a polymeric material (including monomeric or macro-monomeric materials), as used herein, refers to the number-average molecular weight as measured by ¹H NMR spectroscopy unless otherwise specifically noted or unless testing conditions indicate otherwise.

### Compositions, Devices, and Methods

Disclosed herein are hydrogel structures and methods of making and use thereof.

For example, disclosed herein are methods of making a device comprising a hydrogel matrix and a first chamber in the hydrogel matrix, the hydrogel matrix being derived from a prepolymer and the first chamber being perfusable. The methods can, for example, comprise blocking a first portion of a pre-polymerization solution with a first photomask, such that the pre-polymerization solution comprises an exposed portion and a first blocked portion. The pre-polymerization solution can comprise the prepolymer. The methods can further comprise irradiating the exposed portion of the pre-polymerization solution and the first photomask with electromagnetic radiation, the first photomask being substantially opaque to the electromagnetic radiation. The prepolymer within the exposed portion of pre-polymerization solution photopolymerizes to form the hydrogel matrix and the prepolymer within the first blocked portion does not photopolymerize and forms the first chamber.

Also disclosed herein are methods of making a device comprising a hydrogel matrix derived from a prepolymer; a first chamber in the hydrogel matrix, the first chamber being perfusable; and a second chamber in the hydrogel matrix, the second chamber being perfusable and fluidly independent from the first chamber. The methods can, for example, comprise blocking a first portion of a pre-polymerization solution with a first photomask and a second portion of the pre-polymerization solution with a second photomask, the pre-polymerization solution comprising the prepolymer, such that the pre-polymerization solution comprises an exposed portion, a first blocked portion, and a second blocked portion. The methods can further comprise irradiating the exposed portion of the pre-polymerization solution, the first photomask, and the second photomask with electromagnetic radiation, the first photomask and the second photomask being substantially opaque to the electromagnetic radiation. The prepolymer within the exposed portion of pre-polymerization solution photopolymerizes to form the hydrogel matrix, the prepolymer within the first blocked portion does not photopolymerize and forms the first chamber, and the prepolymer within the second blocked portion does not photopolymerize and forms the second chamber.

As used herein, a “chamber” generally refers to a volume that is at least partially enclosed, and in some instances fully enclosed, by the hydrogel matrix. A chamber can, for example, be hollow. In some examples, a chamber can be at least partially filled with a substance.

In some examples, the first chamber can be a first elongated chamber. In some examples, the first chamber can form a first continuous channel within the hydrogel matrix. In some examples, the first continuous channel can be branched.

In some examples, the second chamber can be a second elongated chamber. In some examples, the second chamber can form a second continuous channel within the hydrogel matrix. In some examples, the second continuous channel can be branched.

In some examples, the methods can further comprise, after irradiation, removing the first photomask and the second photomask (when present).

In some examples, the methods can further comprise, after irradiation, rinsing the hydrogel device to remove any remaining pre-polymerization solution and/or prepolymer.

In some examples, the methods can further comprise disposing the pre-polymerization solution in a mold defining a shape before blocking the first portion of the pre-polymerization solution with the first photomask.

The hydrogel matrix can comprise any suitable hydrogel matrix. In some examples, the hydrogel matrix can be selected based on the intended use of the device, the desired properties of the hydrogel matrix, or a combination thereof.

In some examples, the hydrogel matrix comprises a synthetic hydrogel. The synthetic hydrogels can, for example, include a network of crosslinked hydrophilic polymer. Suitable hydrophilic polymers include polyalkylene glycol polymers, polyalkylene oxide homopolymers such as polypropylene glycols, polyoxyethylenated polyols, copolymers thereof and block copolymers thereof, as well as poly(oxyethylated polyol), poly(olefinic alcohol), poly(vinylpyrrolidone), poly(hydroxypropylmethacrylamide), poly(α-hydroxy acid), poly(vinyl alcohol), polyphosphazene, polyoxazoline, poly(N-acryloylmorpholine) and copolymers, terpolymers, and mixtures thereof.

The prepolymer can comprise any suitable prepolymer. In some examples, the prepolymer can be selected based on the intended use of the device, the desired properties of the hydrogel matrix, or a combination thereof.

The term prepolymer is used herein to refer to a polymer that has reactive groups that are available for bond forming reactions that will crosslink (intermolecular and/or intramolecular crosslink). It is not meant to imply that the prepolymer is not yet a polymer (e.g., a monomer or polymer precursor). Rather, a “prepolymer” refers to a starting polymer which contains multiple crosslinkable groups and can be cured (e.g., crosslinked) to obtain a crosslinked polymer having a molecular weight higher than the starting polymer.

The prepolymer has reactive groups that are available for bond formation; that is, the prepolymer can be crosslinked when the reactive groups on separate prepolymers or on the same prepolymer form a bond with the reactive groups of a crosslinker, such as a multifunctional crosslinker.

Examples of reactive groups on a suitable prepolymer include nucleophilic groups or electrophilic groups. Specific examples of nucleophilic reactive groups include thiols (sulfide), amines, azides, nitrites, alcohols (alkoxide), peroxides, carboxylic acids (carboxylate), thiocarboxylic acids (thiocarbonate), sulfonic acids (sulfoxide), and phosphonic acids (phosphates), where the deprotonated form of the reactive group is noted in parenthesis. Enolates can also be suitable nucleophilic reactive groups. Specific examples of electrophilic reactive groups can comprise ketones, aldehydes, alkenes, acyl halides, acrylates, carboxylic acids, esters, hemiacetal, acetals, hemiketal, ketal, orthoesters, amides, imines, imides, azo compounds, cyanates, thiocyanates, nitrates, nitriles, nitrites, thials, phosphines, and phosphodiesters. Other suitable reactive groups can be unsaturated moieties, e.g., an alkene, alkyne, diene, nitrile, azide, carbonyl, or imine.

In some examples, the prepolymer comprises photosensitive groups, such as photosensitive end-groups.

In some examples, the prepolymer comprises a photosensitive prepolymer. The photosensitive prepolymer can comprise any suitable material. For example, the photosensitive prepolymer can comprise poly(ethylene glycol) diacrylate (PEGDA), poly(ethylene glycol) dimethacrylate (PEGDMA), poly(ethylene glycol) diacrylamide (PEGDAAm), gelatin methacrylate (GelMA), collagen methacrylate, silk methacrylate, hyaluronic acid methacrylate, chondroitin sulfate methacrylate, elastin methacrylate, cellulose acrylate, dextran methacrylate, heparin methacrylate, NIPAAm methacrylate, Chitosan methacrylate, polyethylene glycol norbornene, polyethylene glycol dithiol, thiolated gelatin, thiolated chitosan, thiolated silk, PEG based peptide conjugates, cell-adhesive poly(ethylene glycol), MMP-sensitive poly(ethylene glycol), PEGylated fibrinogen, PEGylated collagen, PEGylated laminin, or a combination thereof.

In some examples, the hydrogel matrix is derived from a prepolymer comprising polyethylene glycol or a derivative thereof. In some examples, the hydrogel matrix is derived from a prepolymer comprising poly(ethylene glycol) acrylate, poly(ethylene glycol) diacrylate (PEGDA), poly(ethylene glycol) dimethacrylate (PEGDMA), poly(ethylene glycol) diacrylamide (PEGDAAm), polyethylene glycol norbornene, polyethylene glycol dithiol, PEG based peptide conjugates, cell-adhesive poly(ethylene glycol), MMP-sensitive poly(ethylene glycol). PEGylated fibrinogen, PEGylated collagen, PEGylated laminin, or a combination thereof. In some examples, the hydrogel matrix is derived from a prepolymer comprising poly(ethylene glycol) vinyl sulfone, poly(ethylene glycol) acrylate, poly(ethylene glycol) maleimide, poly(ethylene glycol) norbornene, PEGylated fibrinogen, PEGylated collagen, PEGylated laminin, or a combination thereof. In some examples, the hydrogel matrix is derived from a prepolymer comprising poly(ethylene glycol) vinyl sulfone, poly(ethylene glycol) acrylate, poly(ethylene glycol) maleimide, poly(ethylene glycol)norbornene, or a combination thereof. In some examples, the hydrogel matrix is derived from a prepolymer comprising poly(ethylene glycol) maleimide, poly(ethylene glycol) norbornene, or a combination thereof. In some examples, the hydrogel matrix is derived from a prepolymer comprising poly(ethylene glycol) norbornene.

In some examples, the hydrogel matrix can be derived from a prepolymer having a molecular weight of 0.5 kilodaltons (kDa) or more (e.g., 1 kDa or more, 1.5 kDa or more, 2 kDa or more, 2.5 kDa or more, 3 kDa or more, 3.5 kDa or more, 4 kDa or more, 4.5 kDa or more, 5 kDa or more, 6 kDa or more, 7 kDa or more, 8 kDa or more, 9 kDa or more, 10 kDa or more, 15 kDa or more, 20 kDa or more, 25 kDa or more, 30 kDa or more, 35 kDa or more, 40 kDa or more, 45 kDa or more, 50 kDa or more, 60 kDa or more, 70 kDa or more, 80 kDa or more, 90 kDa or more, 100 kDa or more, 110 kDa or more, 120 kDa or more, 130 kDa or more, 140 kDa or more, 150 kDa or more, 160 kDa or more, 170 kDa or more, 180 kDa or more, or 190 kDa or more). In some examples, the prepolymer can have a molecular weight of 200 kDa or less (e.g., 190 kDa or less, 180 kDa or less, 170 kDa or less, 160 kDa or less, 150 kDa or less, 140 kDa or less, 130 kDa or less, 120 kDa or less, 110 kDa or less, 100 kDa or less, 90 kDa or less, 80 kDa or less, 70 kDa or less, 60 kDa or less, 50 kDa or less, 45 kDa or less, 40 kDa or less, 35 kDa or less, 30 kDa or less, 25 kDa or less, 20 kDa or less, 15 kDa or less, 10 kDa or less, 9 kDa or less, 8 kDa or less, 7 kDa or less, 6 kDa or less, 5 kDa or less, 4.5 kDa or less, 4 kDa or less, 3.5 kDa or less, 3 kDa or less, 2.5 kDa or less, 2 kDa or less, 1.5 kDa or less, or 1 kDa or less). The molecular weight of the prepolymer can range from any of the minimum values described above to any of the maximum values described above. For example, the prepolymer can have a molecular weight of from 0.5 to 200 kilodaltons (kDa)(e.g., from 0.5 to 100 kDa, from 100 to 200 kDa, from 0.5 to 50 kDa, from 50 to 100 kDa, from 100 to 150 kDa, from 150 to 200 kDa, from 0.5 to 175 kDa, from 0.5 to 150 kDa, from 0.5 to 125 kDa, from 0.5 to 75 kDa, from 0.5 to 25 kDa, from 0.5 to 10 kDa, from 1 to 200 kDa, from 2.5 to 200 kDa, from 5 to 200 kDa, from 10 to 200 kDa, from 25 to 200 kDa, from 50 to 200 kDa, from 75 to 200 kDa, from 125 to 200 kDa, from 1 to 175 kDa, from 1 to 150 kDa, from 2 to 150 kDa, from 5 to 100 kDa, from 10 to 100 kDa, from 20 to 100 kDa, from 20 to 80 kDa, from 20 to 60 kDa, from 1 kDa to 50 kDa, from 5 to 50 kDa, from 10 to 50 kDa, from 2 kDa to 40 kDa, from 20 kDa to 40 kDa, from 40 kDa to 60 kDa, from 2 kDa to 25 kDa, or from 2 kDa to 10 kDa).

In some examples, the prepolymer can comprise a branched or multi-arm prepolymer. As used herein, a multi-arm prepolymer describes a prepolymer having a central core with at least two prepolymers covalently attached thereto. Generally, all of the prepolymers attached to the core are the same, but in some instances different prepolymers can be used. Multi-arm prepolymers can have 2 or more arms (e.g., 3 or more, 4 or more, 5 or more, 6 or more, 7 or more, 8 or more, 9 or, or 10 or more). For example, the prepolymer can comprise a branched prepolymer having 2 or more branches (e.g., 3 or more branches, 4 or more branches, 5 or more branches, 6 or more branches, 7 or more branches, 8 or more branches, 9 or more branches, or 10 or more branches).

In some examples, the pre-polymerization solution comprises the prepolymer in an amount of 1 wt. % or more (e.g., 2 wt. % or more, 3 wt. % or more, 4 wt. % or more, 5 wt. % or more, 6 wt. % or more, 7 wt. % or more, 8 wt. % or more, 9 wt. % or more, 10 wt. % or more, 11 wt. % or more, 12 wt. % or more, 13 wt. % or more, 14 wt. % or more, 15 wt. % or more, 16 wt. % or more, 17 wt. % or more, 18 wt. % or more, 19 wt. % or more, 20 wt. % or more, 21 wt. % or more, 22 wt. % or more, 23 wt. % or more, 24 wt. % or more, 25 wt. % or more, 26 wt. % or more, 27 wt. % or more, 28 wt. % or more, or 29 wt. % or more). In some examples, the pre-polymerization solution comprises the prepolymer in an amount of 30 wt. % or less (e.g., 29 wt. % or less, 28 wt. % or less, 27 wt. % or less, 26 wt. % or less, 25 wt. % or less, 24 wt. % or less, 23 wt. % or less, 22 wt. % or less, 21 wt. % or less, 20 wt. % or less, 19 wt. % or less, 18 wt. % or less, 17 wt. % or less, 16 wt. % or less, 15 wt. % or less, 14 wt. % or less, 13 wt. % or less, 12 wt. % or less, 11 wt. % or less, 10 wt. % or less, 9 wt. % or less, 8 wt. % or less, 7 wt. % or less, 6 wt. % or less, 5 wt. % or less, 4 wt. % or less, 3 wt. % or less, or 2 wt. % or less). The amount of prepolymer in the pre-polymerization solution can range from any of the minimum values described above to any of the maximum values described above. For example, the pre-polymerization solution can comprise the prepolymer in an amount of from 1 wt. % to 30 wt. % (e.g., from 1 wt. % to 15 wt. %, from 15 wt. % to 30 wt. %, from 1 wt. % to 5 wt. %, from 5 wt. % to 10 wt. %, from 10 wt. % to 15 wt. %, from 15 wt. % to 20 wt. %, from 20 wt. % to 25 wt. %, from 25 wt. % to 30 wt. %, from 1 wt. % to 25 wt. %, from 1 wt. % to 20 wt. %, from 1 wt. % to 10 wt. %, from 5 wt. % to 30 wt. %, from 10 wt. % to 30 wt. %, from 20 wt. % to 30 wt. %, from 2 wt. % to 29 wt. %, from 5 wt. % to 25 wt. %, from 10 wt. % to 20 wt. %, or from 4 wt. % to 10 wt. %).

In some examples, pre-polymerization solution further comprises a crosslinker. In some examples, the hydrogel is further derived from the crosslinker. The crosslinker can be any suitable crosslinker. In some examples, the crosslinker can be selected in view of the prepolymer, the intended use of the device, the desired properties of the hydrogel matrix, or a combination thereof.

In some examples, the crosslinker is a multifunctional crosslinker. The multifunctional crosslinker has reactive groups that are available for bond formation; that is the multifunctional crosslinker can be crosslinked when the reactive groups of the prepolymer. Examples of reactive groups on a suitable multifunctional crosslinker include nucleophilic groups or electrophilic groups. The reactive groups of the multifunctional crosslinker can be complementary to the reactive groups of the prepolymer. For example, if the reactive groups of the prepolymer comprise electrophilic reactive groups the multifunctional crosslinker can comprise nucleophilic reactive groups.

In some examples, the multifunctional crosslinker can comprise 2 or more reactive groups (e.g., 3 or more, 4 or more, or S or more). In some examples the multifunctional crosslinker can comprise 6 or less reactive groups (e.g., 5 or less, 4 or less, or 3 or less). The number of reactive groups of the multifunctional crosslinker can range from any of the minimum values described above to any of the maximum values described above, for example from 2 to 6 (e.g., from 2 to 4, from 4 to 6, from 3 to 5, from 2 to 3, from 3 to 4, from 4 to 5, or from 5 to 6).

In some examples, the multifunctional crosslinker can comprise a multifunctional thiol. In some examples, the crosslinker comprises a dithiol.

In some examples, the prepolymer comprises poly(ethylene glycol) norbornene, such as a branched or multi-armed poly(ethylene glycol) norbornene, and the crosslinker comprises a thiol, such as a multifunctional thiol. For example, the photopolymerization can comprise thiol-norbornene photopolymerization.

In some examples, the hydrogel matrix is further derived from one or more additional components, such as one or more additional monomers, prepolymers, ligands, chemical agents, therapeutic agents, photoinitiators, or a combination thereof. In some examples, the pre-polymerization solution further comprises said one or more additional components.

In some examples, the hydrogel matrix is further derived from a natural or biological component, such as a natural or biological prepolymer. In some examples, the natural or biological prepolymer comprises fibrinogen, collagen, or a combination thereof.

In some examples, the pre-polymerization solution further comprises a photoinitiator.

In some example, the hydrogel matrix further comprises a chemical agent, a therapeutic agent, or a combination thereof dispersed therein. In some examples, the pre-polymerization solution further comprises the chemical agent and/or the therapeutic agent. The therapeutic agent can, for example, comprise an anticancer agent, anti-inflammatory agent, antimicrobial agent, or a combination thereof. As used herein, antimicrobials include, for example, antibacterials, antifungals, and antivirals.

In some examples, the chemical agent and/or the therapeutic agent is/are dispersed inhomogeneously within the hydrogel matrix. For example, the chemical agent and/or therapeutic agent can have a concentration that varies across the hydrogel matrix, such that the chemical agent and/or the therapeutic agent has a compositional gradient across the hydrogel matrix. The compositional gradient can, for example, be a linear gradient, a stepped gradient, an exponential gradient, a logarithmic gradient, etc., or a combination thereof.

The electromagnetic radiation can comprise any suitable electromagnetic radiation. In some examples, the electromagnetic radiation can be selected in view of the crosslinker, the prepolymer, the intended use of the device, the desired properties of the hydrogel matrix, or a combination thereof.

In some examples, the electromagnetic radiation can comprise light. In some examples, the electromagnetic radiation comprises UV radiation.

In some examples, the electromagnetic radiation comprises one or more wavelengths of 10 nanometers (nm) or more (e.g., 15 nm or more, 20 nm or more, 25 nm or more, 30 nm or more, 35 nm or more, 40 nm or more, 45 nm or more, 50 μm or more, 60 nm or more, 70 nm or more, 80 nm or more, 90 nm or more, 100 nm or more, 125 nm or more, 150 nm or more, 175 nm or more, 200 nm or more, 225 nm or more, 250 nm or more, 275 nm or more, 300 nm or more, 325 nm or more, 350 nm or more, 375 nm or more, 400 nm or more, 450 nm or more, 500 nm or more, 550 nm or more, 600 nm or more, 650 nm or more, 700 nm or more, 750 nm or more, 800 nm or more, or 850 nm or more). In some examples, the electromagnetic radiation comprises one or more wavelengths of 900 nm or less (e.g., 850 nm or less, 800 nm or less, 750 nm or less, 700 nm or less, 650 nm or less, 600 nm or less, 550 nm or less, 500 nm or less, 450 nm or less, 400 nm or less, 375 nm or less, 350 nm or less, 325 nm or less, 300 nm or less, 275 nm or less, 250 nm or less, 225 nm or less, 200 nm or less, 175 nm or less, 150 nm or less, 125 nm or less, 100 nm or less, 90 nm or less, 80 nm or less, 70 nm or less, 60 nm or less, 50 nm or less, 45 nm or less, 40 nm or less, 35 nm or less, 30 nm or less, 25 nm or less, 20 nm or less, or 15 nm or less). The one or more wavelength(s) of the electromagnetic radiation can range from any of the minimum values described above to any of the maximum values described above. For example, the electromagnetic radiation can comprise one or more wavelengths of from 10 nm to 900 nm (e.g., from 10 nm to 450 nm, from 450 nm to 900 nm, from 10 nm to 300 nm, from 300 nm to 600 nm, from 600 nm to 900 nm, from 10 nm to 800 nm, from 10 nm to 700 nm, from 10 nm to 600 nm, from 10 nm to 500 nm, from 10 nm to 400 nm, from 10 nm to 200 nm, from 10 nm to 100 nm, from 25 nm to 900 nm, from 50 nm to 900 nm, from 100 nm to 900 nm, from 200 nm to 900 nm, from 300 nm to 900 nm, from 400 nm to 900 nm, from 500 nm to 900 nm, from 700 nm to 900 nm, from 800 nm to 900 nm, from 25 nm to 850 nm, from 100 nm to 900 nm, from 100 nm to 400 nm, from 400 nm to 900 nm, or from 400 nm to 750 nm).

In some examples, the electromagnetic radiation is provided by a light source. The light source can be any type of light source. Examples of suitable light sources include natural light sources (e.g., sunlight) and artificial light sources (e.g., incandescent light bulbs, light emitting diodes, gas discharge lamps, arc lamps, lasers, etc.). In some examples, the light source is an artificial light source. In some examples, the light source comprises a light emitting diode (LED), a lamp, a laser, or a combination thereof.

The exposed portion of the pre-polymerization solution, the first photomask, and the second photomask (when present) can be irradiated for an amount of time of 1 millisecond or more (e.g., 2 milliseconds or more, 3 milliseconds or more, 4 milliseconds or more, 5 milliseconds or more, 10 milliseconds or more, 15 milliseconds or more, 20 milliseconds or more, 25 milliseconds or more, 30 milliseconds or more, 35 milliseconds or more, 40 milliseconds or more, 50 milliseconds or more, 60 milliseconds or more, 70 milliseconds or more, 80 milliseconds or more, 90 milliseconds or more, 100 milliseconds or more, 125 milliseconds or more, 150 milliseconds or more, 175 milliseconds or more, 200 milliseconds or more, 225 milliseconds or more, 250 milliseconds or more, 300 milliseconds or more, 350 milliseconds or more, 400 milliseconds or more, 450 milliseconds or more, 500 milliseconds or more, 600 milliseconds or more, 700 milliseconds or more, 800 milliseconds or more, 900 milliseconds or more, 1 second or more, 2 seconds or more, 3 seconds or more, 4 seconds or more, 5 seconds or more, 10 seconds or more, 15 seconds or more, 20 seconds or more, 25 seconds or more, 30 seconds or more, 35 seconds or more, 40 seconds or more, 45 seconds or more, 50 seconds or more, 55 seconds or more, 1 minute or more, 2 minutes or more, 3 minutes or more, 4 minutes or more, 5 minutes or more, 10 minutes or more, 15 minutes or more, 20 minutes or more, 25 minutes or more, 30 minutes or more, 35 minutes or more, 40 minutes or more, 45 minutes or more, 50 minutes or more, or 55 minutes or more). In some examples, the exposed portion of the pre-polymerization solution, the first photomask, and the second photomask (when present) can be irradiated for an amount of time of 1 hour or less (e.g., 55 minutes or less, 50 minutes or less, 45 minutes or less, 40 minutes or less, 35 minutes or less, 30 minutes or less, 25 minutes or less, 20 minutes or less, 15 minutes or less, 10 minutes or less, 5 minutes or less, 4 minutes or less, 3 minutes or less, 2 minutes or less, 1 minute or less, 55 seconds or less, 50 seconds or less, 45 seconds or less, 40 seconds or less, 35 seconds or less, 30 seconds or less, 25 seconds or less, 20 seconds or less, 15 seconds or less, 10 seconds or less, 5 seconds or less, 4 seconds or less, 3 seconds or less, 2 seconds or less, 1 second or less, 900 milliseconds or less, 800 milliseconds or less, 700 milliseconds or less, 600 milliseconds or less, 500 milliseconds or less, 450 milliseconds or less, 400 milliseconds or less, 350 milliseconds or less, 300 milliseconds or less, 250 milliseconds or less, 225 milliseconds or less, 200 milliseconds or less, 175 milliseconds or less, 150 milliseconds or less, 125 milliseconds or less, 100 milliseconds or less, 90 milliseconds or less, 80 milliseconds or less, 70 milliseconds or less, 60 milliseconds or less, 50 milliseconds or less, 40 milliseconds or less, 35 milliseconds or less, 30 milliseconds or less, 25 milliseconds or less, 20 milliseconds or less, 15 milliseconds or less, 10 milliseconds or less, 5 milliseconds or less, 4 milliseconds or less, 3 milliseconds or less, or 2 milliseconds or less). The amount of time that the exposed portion of the pre-polymerization solution, the first photomask, and the second photomask (when present) are irradiated can range from any of the minimum values described above to any of the maximum values described above. For example, the exposed portion of the pre-polymerization solution, the first photomask, and the second photomask (when present) can be irradiated for an amount of time of from 1 millisecond to 1 hour (e.g., from 1 millisecond to 1 second, from 1 second to 1 minute, from 1 minute to 1 hour, from 1 millisecond to 30 minutes, from 1 millisecond to 15 minutes, from 1 millisecond to 10 minutes, from 1 millisecond to 5 minutes, from 1 millisecond to 1 minute, from 1 millisecond to 30 seconds, from 1 millisecond to 15 seconds, from 1 millisecond to 10 seconds, or from 1 millisecond to 5 seconds).

In some examples, the hydrogel matrix exhibits a swelling of 10% or less (e.g., 9% or less, 8% or less, 7% or less, 6% or less, 5% or less, 4% or less, 3% or less, 2% or less, or 1% or less) in physiological saline. The swelling can, for example, be determined based on weight change of the hydrogel before and after being exposed to or soaked in physiological saline.

In some examples, the hydrogel matrix exhibits a shape fidelity of 50% or more (e.g., 55% or more, 60% or more, 65% or more, 70% or more, 75% or more, 80% or more, 85% or more, 90% or more, 95% or more, or 99% or more). The shape fidelity can be measured based on optical changes in the geometry of features of the hydrogel matrix before and after being exposed to or soaked in a solvent, such as physiological saline.

In some examples, the hydrogel matrix has a storage modulus of greater than 0 Pa (e.g., 1 Pa or more, 2 Pa or more, 3 Pa or more, 4 Pa or more, 5 Pa or more, 10 Pa or more, 15 Pa or more, 20 Pa or more, 25 Pa or more, 30 Pa or more, 35 Pa or more, 40 Pa or more, 45 Pa or more, 50 Pa or more, 60 Pa or more, 70 Pa or more, 80 Pa or more, 90 Pa or more, 100 Pa or more, 125 Pa or more, 150 Pa or more, 175 Pa or more, 200 Pa or more, 225 Pa or more, 250 Pa or more, 300 Pa or more, 350 Pa or more, 400 Pa or more, 450 Pa or more, 500 Pa or more, 550 Pa or more, 600 Pa or more, 700 Pa or more, 800 Pa or more, 900 Pa or more, 1000 Pa or more, 1250 Pa or more, 1500 Pa or more, 1750 Pa or more, 2000 Pa or more, 2250 Pa or more, 2500 Pa or more, 3000 Pa or more, 3500 Pa or more, 4000 Pa or more, or 4500 Pa or more). In some examples, the hydrogel matrix has a storage modulus of 5000 Pa or less (e.g., 4500 Pa or less, 4000 Pa or less, 3500 Pa or less, 3000 Pa or less, 2500 Pa or less, 2250 Pa or less, 2000 Pa or less, 1750 Pa or less, 1500 Pa or less, 1250 Pa or less, 1000 Pa or less, 900 Pa or less, 800 Pa or less, 700 Pa or less, 600 Pa or less, 550 Pa or less, 500 Pa or less, 450 Pa or less, 400 Pa or less, 350 Pa or less, 300 Pa or less, 250 Pa or less, 225 Pa or less, 200 Pa or less, 175 Pa or less, 150 Pa or less, 125 Pa or less, 100 Pa or less, 90 Pa or less, 80 Pa or less, 70 Pa or less, 60 Pa or less, 50 Pa or less, 45 Pa or less, 40 Pa or less, 35 Pa or less, 30 Pa or less, 25 Pa or less, 20 Pa or less, 15 Pa or less, 10 Pa or less, 5 Pa or less, 4 Pa or less, 3 Pa or less, 2 Pa or less, or 1 Pa or less). The storage modulus of the hydrogel matrix can range from any of the minimum values described above to any of the maximum values described above. For example, the hydrogel matrix can have a storage modulus of from greater than 0 Pa to 5000 Pa (e.g., from greater than 0 to 2500 Pa, from 2500 to 5000 Pa, from greater than 0 to 1000 Pa, from 1000 to 2000 Pa, from 2000 to 3000 Pa, from 3000 to 4000 Pa, from 4000 to 5000 Pa, from greater than 0 to 4000 Pa, from greater than 0 to 3000 Pa, from greater than 0 to 2000 Pa, from greater than 0 to 800 Pa, from greater than 0 to 600 Pa, from greater than 0 to 500 Pa, from greater than 0 to 400 Pa, from greater than 0 to 300 Pa, from greater than 0 to 200 Pa, from greater than 0 to 100 Pa, from greater than 0 to 50 Pa, from greater than 0 to 25 Pa, from greater than 0 to 10 Pa, from 1 to 5000 Pa, from 5 to 5000 Pa, from 10 to 5000 Pa, from 25 to 5000 Pa, from 50 to 5000 Pa, from 100 to 5000 Pa, from 200 to 5000 Pa, from 300 to 5000 Pa, from 400 to 5000 Pa, from 500 to 5000 Pa, from 600 to 5000 Pa, from 800 to 5000 Pa, from 2000 to 5000 Pa, from 3000 to 5000 Pa, from 5 to 4500 Pa, from 10 to 4000 Pa, or from 10 to 1000 Pa).

The hydrogel matrix can, for example, be stable for an amount of time. For example, the hydrogel can be stable for an amount of time after being exposed to or soaked in a solvent, such as physiological saline. As used herein, “stable” means that 10 wt. % or less (e.g., 9% or less, 8% or less, 7% or less, 6% or less, 5% or less, 4% or less, 3% or less, 2% or less, or 1% or less) of the hydrogel matrix degrades over the selected time period.

In some examples, the hydrogel matrix is stable for an amount of time of from 1 day or more (e.g., 2 days or more, 3 days or more, 4 days or more, 5 days or more, 6 days or more, 1 week or more, 1.5 weeks or more, 2 weeks or more, 2.5 weeks or more, 3 weeks or more, 3.5 weeks or more, 1 month or more, 1.5 months or more, 2 months or more, or 2.5 months or more). In some examples, the hydrogel matrix is stable for an amount of time of 3 months or less (e.g., 2.5 months or less, 2 months or less, 1.5 months or less, 1 month or less, 3.5 weeks or less, 3 weeks or less, 2.5 weeks or less, 2 weeks or less, 1.5 weeks or less, 1 week or less, 6 days or less, 5 days or less, 4 days or less, 3 days or less, or 2 days or less). The amount of time that the hydrogel matrix is stable can range from any of the minimum values described above to any of the maximum values described above. For example, the hydrogel matrix can be stable for an amount of time of from 1 day to 3 months (e.g., 1 day to 1.5 months, from 1.5 months to 3 months, from 1 day to 1 week, from 1 week to 1 month, from 1 month to 3 months, from 1 day to 2 months, from 1 day to 1 month, from 1 day to 3 weeks, from 1 day to 2 weeks, from 1 day to 5 days, from 2 days to 3 months, from 3 days to 3 months, from 4 days to 3 months, from 5 days to 3 months, from 6 days to 3 months, from 1 week to 3 months, from 2 weeks to 3 months, from 3 weeks to 3 months, from 2 days to 2.5 months, or from 5 days to 2 months).

In some examples, the hydrogel matrix is continuous. “Continuous,” as used herein, generally refers to a phase such that all points within the phase are directly connected three-dimensionally, so that for any two points within a continuous phase, there exists a path in three-dimensional space which connects the two points without leaving the phase.

In some examples, the hydrogel matrix is monolithic.

In some examples, the hydrogel matrix is porous.

In some examples, the hydrogel matrix is biocompatible.

In some examples, the hydrogel matrix is biodegradable.

In some examples, the hydrogel matrix comprises a photopolymerized polymer network. In some examples, the hydrogel matrix comprises a cross-linked polymer network.

In some examples, the hydrogel matrix comprises a photopolymerized polymer network derived from a photosensitive polymer. In some examples, the hydrogel matrix comprises a cross-linked polymer network derived from a photosensitive polymer.

Also disclosed herein are devices made by any of the methods disclosed herein. For example, also disclosed herein are devices made by the methods disclosed herein, the devices comprising a hydrogel matrix and a first chamber in the hydrogel matrix, the hydrogel matrix being derived from a prepolymer and the first chamber being perfusable. Also disclosed herein are devices made by the methods disclosed herein, the devices comprising a hydrogel matrix derived from a prepolymer; a first chamber in the hydrogel matrix, the first chamber being perfusable; and a second chamber in the hydrogel matrix, the second chamber being perfusable and fluidly independent from the first chamber.

In some examples, the hydrogel matrix, the first chamber, the second chamber, or a combination thereof can have complex geometries, chemical gradients, or patterned cells.

In some examples, the device is a microfluidic device.

In some examples, the hydrogel structure is implantable in a subject.

Also disclosed herein are methods of uses of any of the devices disclosed herein. For example, also disclosed herein are methods of uses of any of the devices made by any of the methods disclosed herein.

The methods can, for example, comprise using the device for diagnostics, disease modeling, regenerative medicine, drug screening, tissue modeling, or a combination thereof.

In some examples, the methods can comprise using the device as a biomaterial substrate or scaffold, a cell culture substrate or platform, or a combination thereof.

In some examples, the method comprises using the device as a biomaterial substrate or scaffold and/or a cell culture substrate or platform for a surface functionalized protein, peptide, biomolecule, or combination thereof.

In some examples, the method comprises seeding the device (e.g., the first and/or second chamber) with a cell and/or biomaterial, and perfusing the device (e.g., the first and/or second chamber) with a solvent or solution. In some examples, the solvent or solution can comprise cell culture media, physiological saline, proteins, peptides, saccharides, ions, nucleic acids (e.g., DNA, RNA), oligonucleotides, metabolites, exosomes, bacteria, viruses, and/or other biological or chemical molecules.

In some examples, the method comprises using the device as a cell culture substrate for any cell, such as an engineered cell. For example, the method can comprise using the device as a cell culture substrate for any cell, such as an engineered cell, that presents proteins or other biomolecules.

In some examples, the method comprises using the device as a cell culture substrate for immunocytes, B cells, lymph cells, dendritic cells, lung cells, intestinal cells, endothelial cells, hepatocytes, kidney epithelial cells, or a combination thereof.

In some examples, the method comprises using the device as a cell culture substrate and the cultured cells exhibit a cell viability of 50% or more (e.g., 55% or more, 60% or more, 65% or more, 70% or more, 75% or more, 80% or more, 85% or more, 90% or more, or 95% or more) after 4 days or more (e.g., 5 days or more, 6 days or more, 1 week or more, 1.5 weeks or more, 2 weeks or more, 2.5 weeks or more, 3 weeks or more, 3.5 weeks or more, 1 month or more, 1.5 months or more, 2 months or more, or 2.5 months or more) in the device.

In some examples, the method comprises using the device as a cell culture substrate and the cultured cells exhibit cell phenotype differentiation.

In some examples, the method comprises using the device as a cell culture substrate and the cultured cells comprise viable intestinal cells displaying appropriate apical and basolateral marker localization, differentiated epithelial cells, or a combination thereof.

In some examples, the method comprises using the device as a cell culture substrate and the cultured cells colonize the device in multiple dimensions (e.g., tri-dimensionally).

In some examples, the method comprises using the device to grow three-dimensional cell clusters.

In some examples, the method comprises using the device to grow an organoid. In some examples, the method comprises using the device to grow a lymphoid follicle organoid, a tonsil organoid, an intestinal organoid, a lung organoid, or a combination thereof.

In some examples, the method comprises using the device to grow a human organoid. In some examples, the method comprises using the device to grow a human intestinal organoid.

Also disclosed herein are articles of manufacture comprising any of the devices disclosed herein. For example, also disclosed herein are articles of manufacture comprising any of the devices made by any of the methods disclosed herein. The article can, for example, comprise a biomaterial substrate or scaffold, a cell culture substrate or platform, or a combination thereof.

A number of embodiments of the invention have been described. Nevertheless, it will be understood that various modifications may be made without departing from the spirit and scope of the invention. Accordingly, other embodiments are within the scope of the following claims.

The examples below are intended to further illustrate certain aspects of the systems and methods described herein and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of measurement conditions, e.g., component concentrations, temperatures, pressures and other measurement ranges and conditions that can be used to optimize the described process.

### Example 1

Disclosed herein is a rapid and facile light-based approach to generate complex hydrogel structures for tissue/organ on a chip models.

In some examples, the methods disclosed herein involve a rapid and facile light-based approach to generate complex hydrogel structures to use in tissue/organ-on-a-chip models for diagnostic, disease modeling, and drug screening applications. Presently, complex tissue on a chip models, such as a perfusable gut-on-a-chip, involve painstaking, time-consuming, and equipment-intensive methods using laser ablation techniques of matrices. This approach has been limited to natural, biological matrices (e.g., Matrigel) that further limits application. The methods disclosed herein employ light-triggered polymerization of synthetic hydrogels and a photomask for generation of complex patterns, including perfusable channels. This approach reduces preparation times from several hours to minutes and uses comparatively simple instrumentation. In addition, the ability to use synthetic hydrogels over natural matrices provides tremendous flexibility and lowers regulatory burdens.

Advantages of the methods disclosed herein include, but are not limited to: rapidity (minutes instead of hours); simplicity (simple light exposure and equipment); and use of synthetic matrices.

### Example 2

Disclosed herein is a rapid and facile method for patterning hydrogels to generate complex scaffolds.

In some examples, the methods disclosed herein involves a rapid and facile approach to generate complex hydrogel structures to use in tissue/organ-on-a-chip models for diagnostic, disease modeling, regenerative medicine, and drug screening applications. Presently, complex tissue models involve painstaking, time-consuming, and equipment-intensive methods using laser-based patterning or ablation of matrices. The strategy disclosed herein employs spatial patterning of multiple cell types and other non-cellular entities in a synthetic hybrid hydrogel system for the generation of complex patterns, including biomolecular and chemical gradients. In this approach, one or more type of cells is encapsulated in a microgel and co-encapsulated in a bulk hydrogel matrix with another set of cells, along with the gel composition designed for tunable degradation and biophysical properties. This approach reduces preparation times from several hours to minutes and requires simple instrumentation. In addition, the ability to use this approach over complex patterning approaches, like laser patterns, provides tremendous flexibility and lowers regulatory burdens

Advantages of the methods disclosed herein include, but are not limited to: rapidity (minutes instead of hours); simplicity (simple light exposure and equipment); and use of synthetic matrices.

### Example 3—Decoding Specificity Between Antibodies and Vaccines Using Integrated Human Lymphoid, Gut, and Lung Organoids

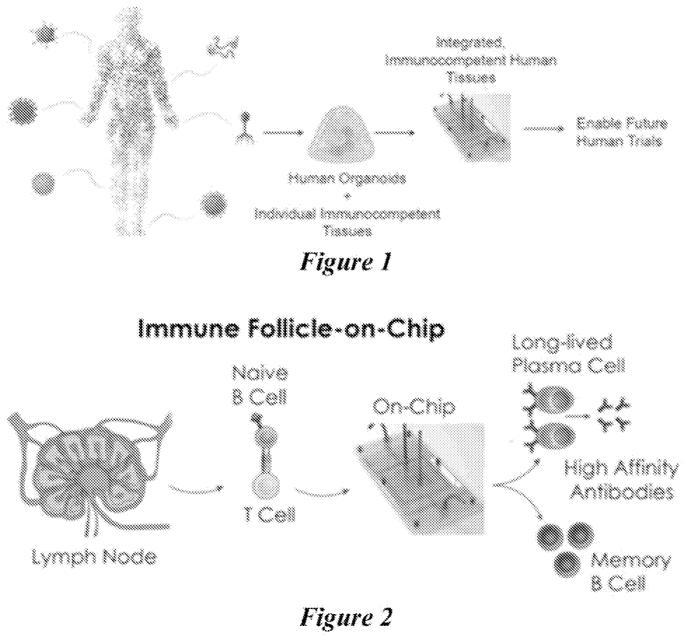

It is desired to engineer a multiorgan platform that recreates and connects lymphatic systems and tissue-resident immunity, and recreate immunological mechanisms of pathologies and drug-induced reaction for interventions targeting cancer, infectious diseases and organ transplantation (FIG. 1).

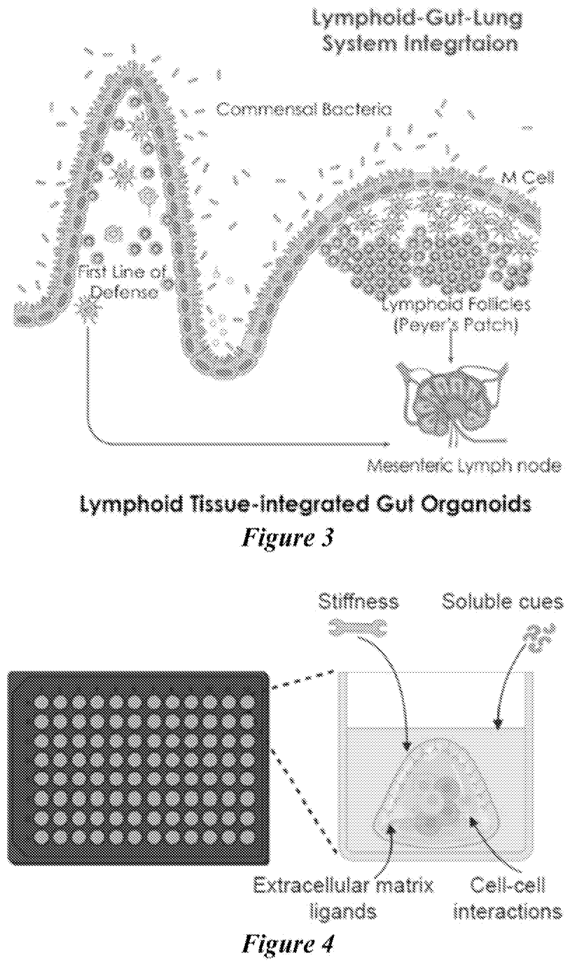

Described herein are compositions, devices, and methods for generating a human lymphoid-on-a-chip system and interconnecting with gut and lung organ model systems to better understand mucosal-lymphoid immune crosstalk during infection and vaccination. Bioinspired immune follicle-on-a-chip can recapitulate germinal center (GC)-like cell populations and functionality (FIG. 2). Lymphoid tissues can be integrated with immunocompetent intestinal organoids (FIG. 3) and other mucosal organ systems, such as lungs.

Follicle-on-a-chip

It is desired to engineer an immune follicle-on-a-chip that recapitulates the phenotype, characteristics, and decision making of human B cells (FIG. 2).

The experimental approach involves defining a lymphoid microenvironment, engineering an immune follicle-on-a-chip, and evaluating B cell activation. The immune follicle-on-a-chip device can be a microfluidic based system comprising hydrogel functionalities that can support B cell processes. The device can comprise a gradient of hydrogel-encapsulated immune and stromal cells. B cell activation can be evaluated using omics, sequencing, biochemical assays, and flow cytometry.

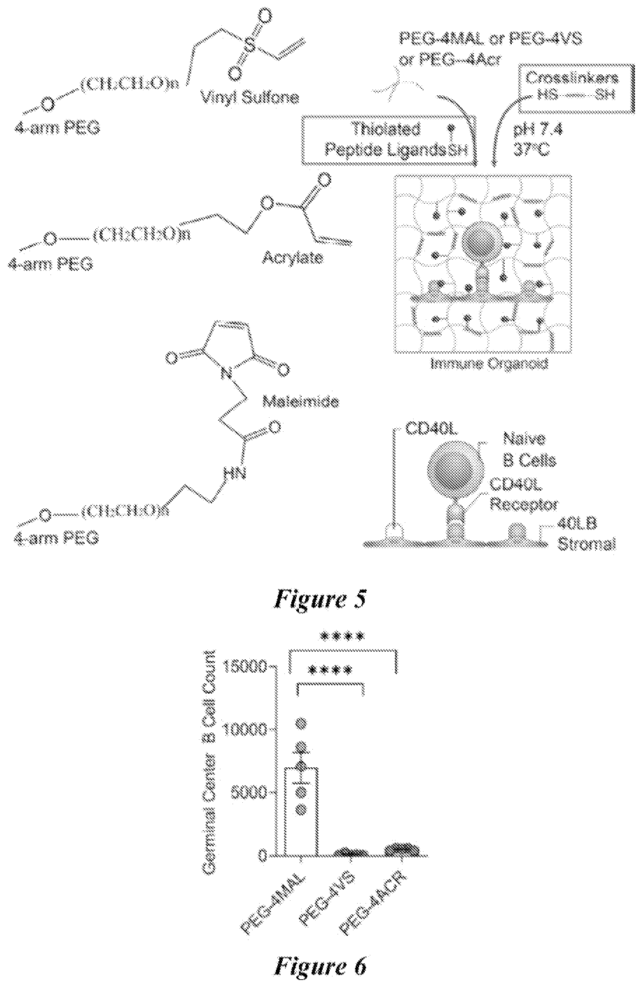

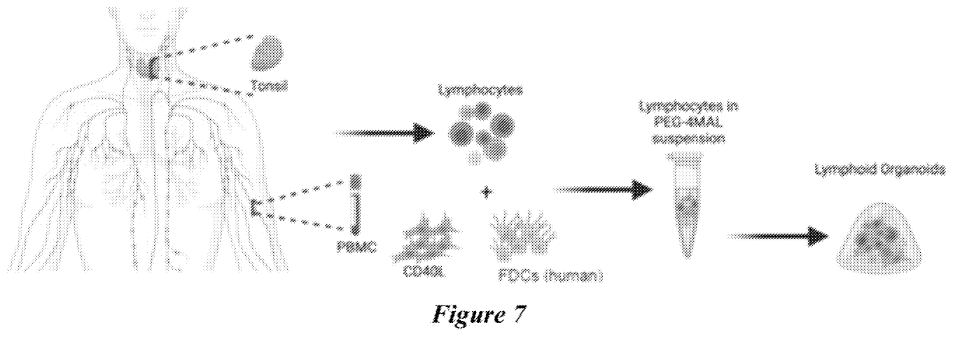

Approach towards biomaterials-based human lymphoid follicle organoids. A schematic diagram of the approach is shown in FIG. 4, FIG. 5 and FIG. 7.

Lymphoid Cell Survival and Activation. Germinal Center response was determined from Tonsil/PBMC in organoids (FIG. 6). The conditions were optimized for Tonsil/PBMC organoids.

Cytokines included IL4 (2d) and IL21 (2d). Cell types were: 1) PBMC, engineered CD40LL, and FDCs (human); and 2) Tonsil, engineered CD40LL, and FDCs (human). Endpoint included D4 and D8 (N=3-5). Formulations included PBS, H1N1 antigen only, TLR7/8 agonist and H1N1, and TLR3 agonist an H1N1.

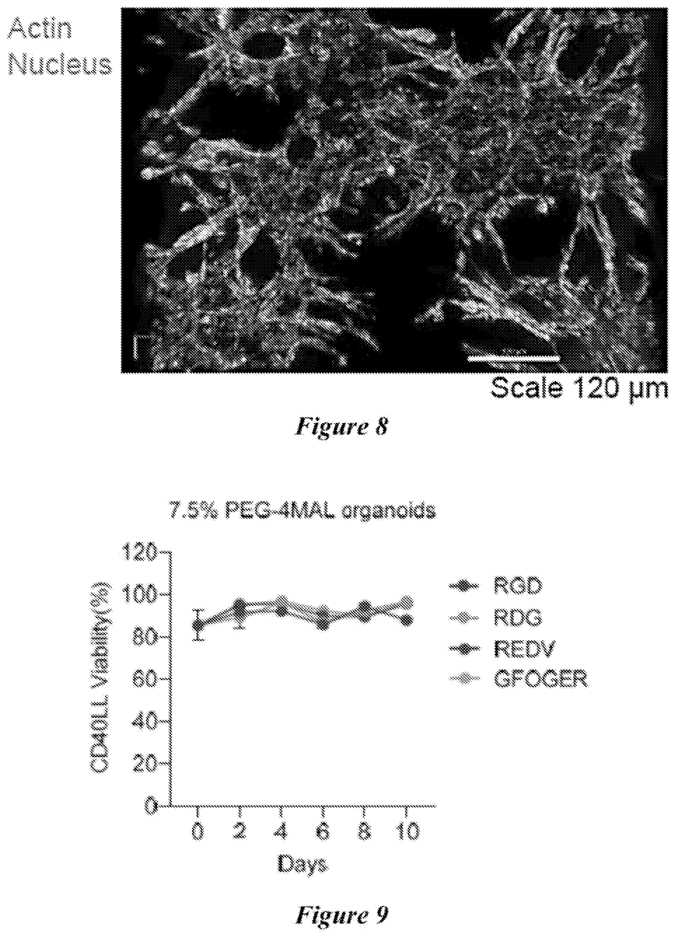

PEG-4MAL Organoid Characterization. The effect of ECM and hydrogel wt. % on PEG-4MAL based organoids was assessed. EMC-dependent/independent spreading and survival of stromal CD40L cells and FDCs was established (FIG. 8-FIG. 9). Temporal stability and impact of polymer weight % on hydrogel stiffness was quantified (FIG. 10).

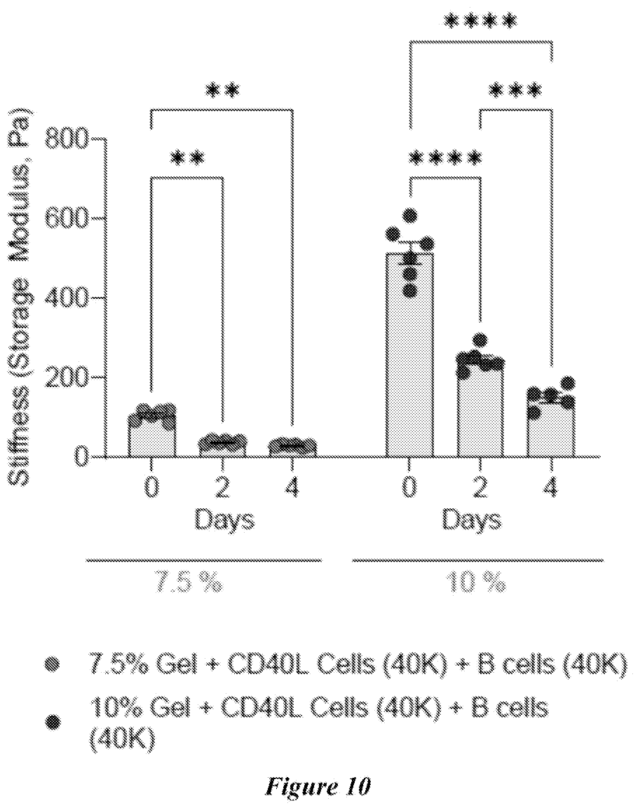

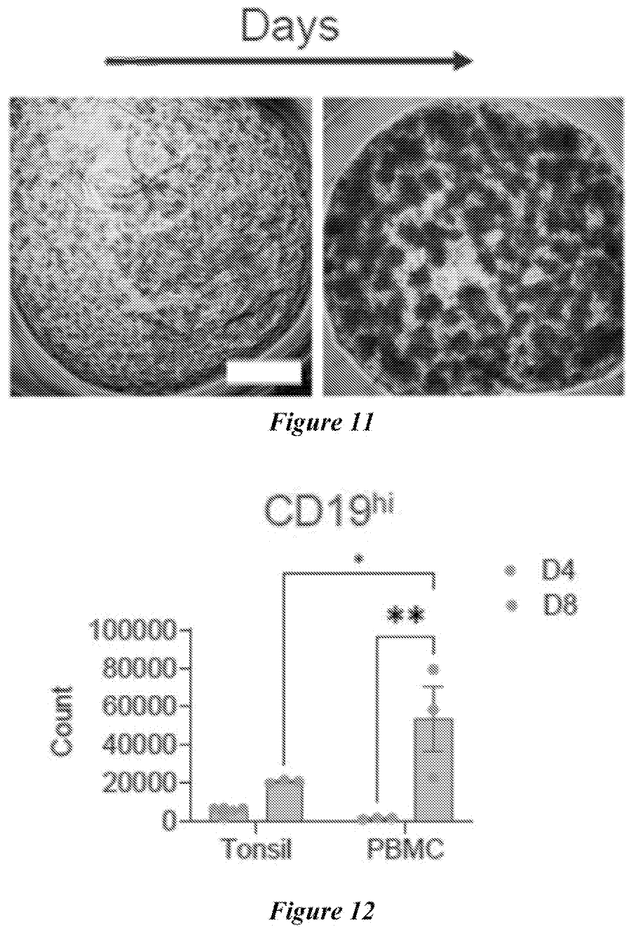

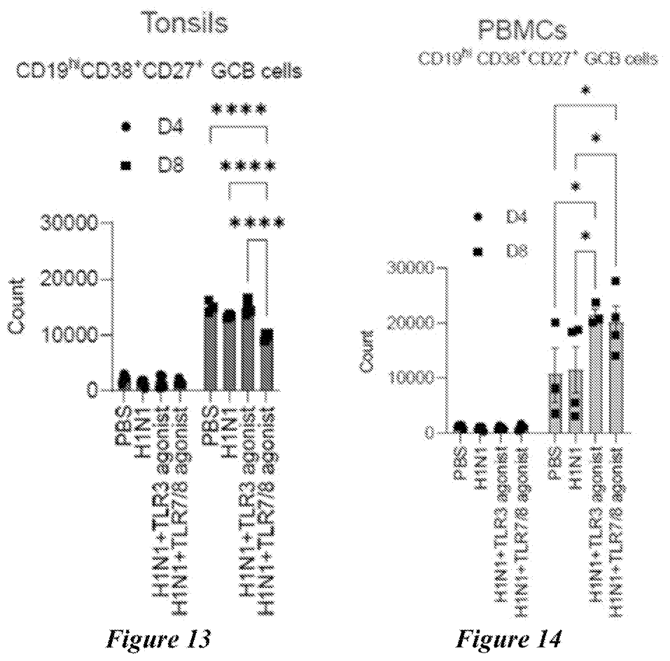

Primary Human B Cell Survival. To assess primary human B cell survival, PBMCs and Tonsils were compared (FIG. 11-FIG. 14). At D8, double the number of CD19^{hi}+ B cells were observed in PBMC-organoids versus Tonsil organoids (FIG. 12). At D8, PBMC-organoids have more GC B cells then Tonsil-organoids (FIG. 13-FIG. 14). At D8, PDMC-organoids show effect of TLR agonists (FIG. 13-FIG. 14).

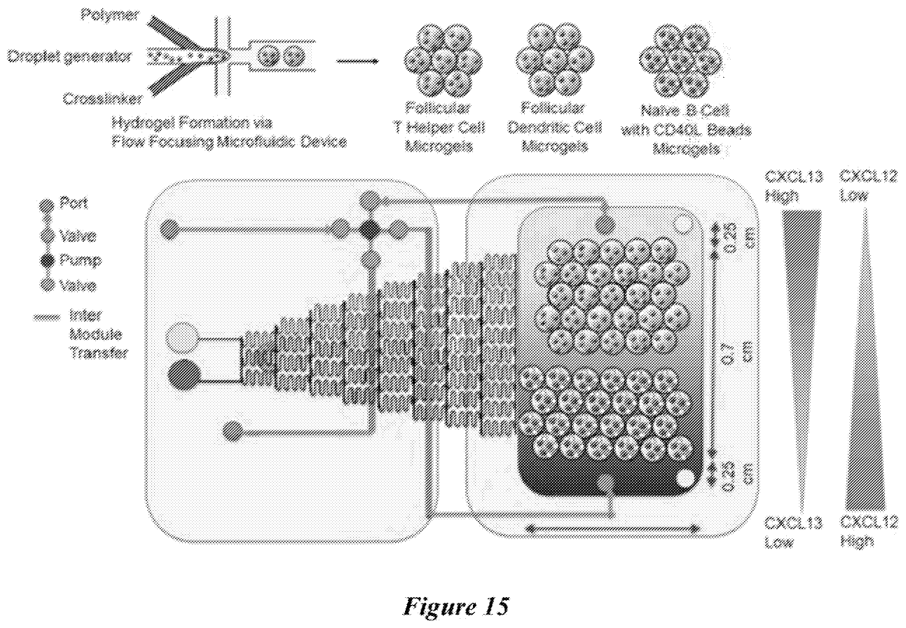

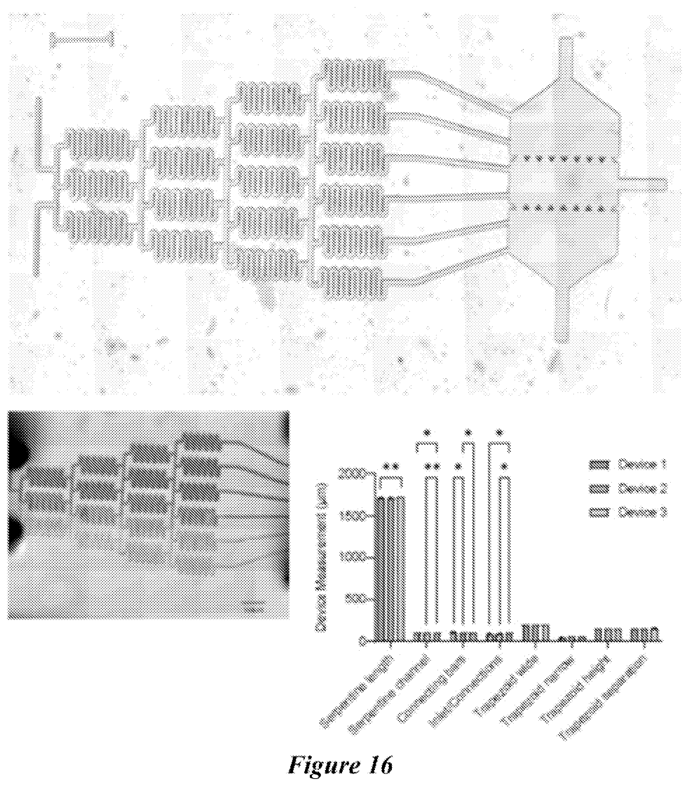

Lymphoid Follicle-on-a-chip Engineering—PEG-4MAL. For lymphoid follicle-on-a-chip engineering, it was desired to develop a microfluidic set up with high fidelity, e.g. >85% of desired features retained (FIG. 15-FIG. 16). A challenge was that PEG-4MAL macromer gels quickly and therefore may not be suitable for inclusion in large scale devices.

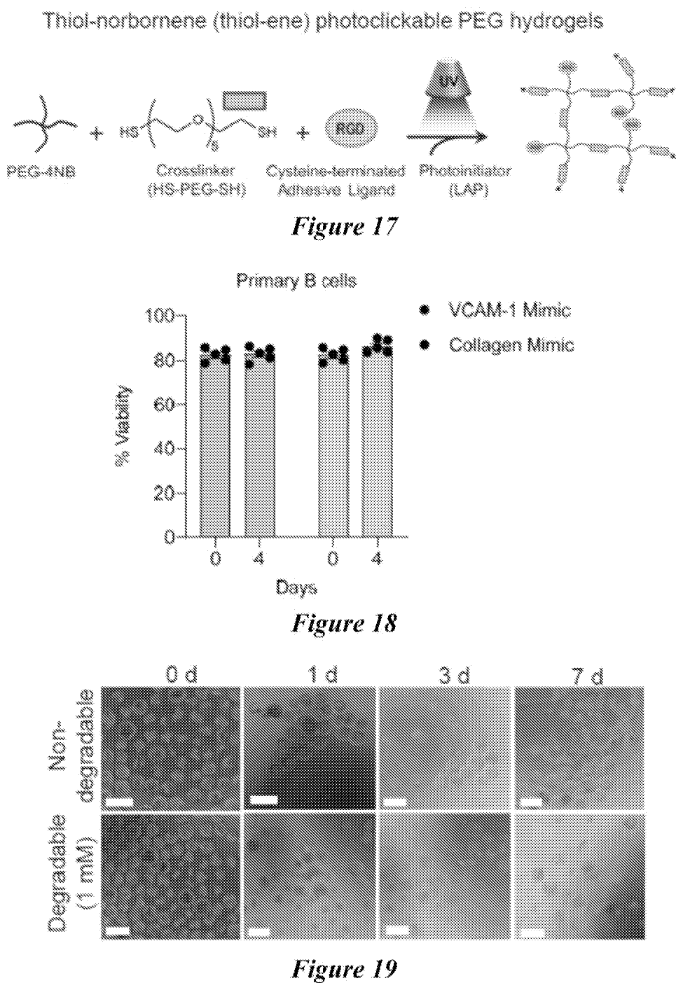

Bioengineered gradient devices for lymphoid follicles on-a-chip. Due to the above challenges with PEG-4MAL, thiol-norbornene (thiol-ene) photoclickable PEG hydrogels were investigated (FIG. 17). These compositions were found to gel within 10-15 seconds of UV exposure. Unlike PEG-vinyl sulfones and acrylates, the thiol-norbornene bioconjugate chemistry was found to be amendable to high survival of primary B cells, comparable to PEG maleimide (FIG. 18).

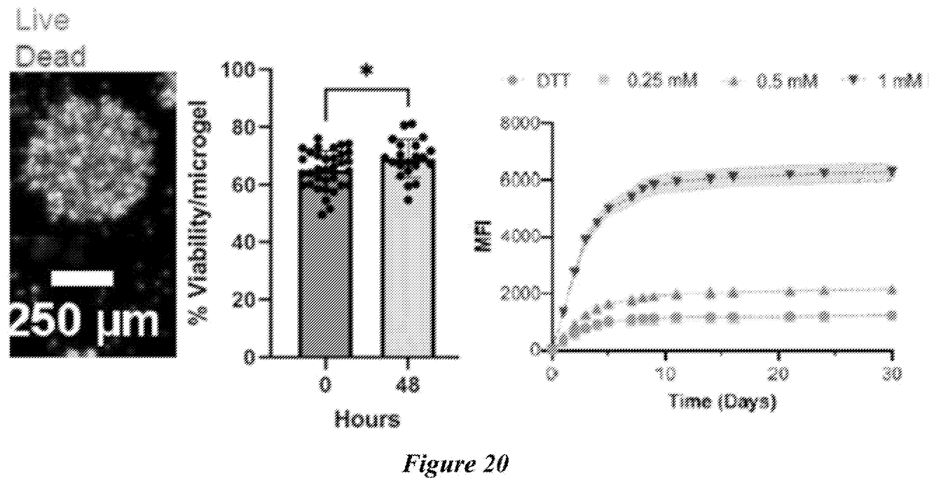

Lymphoid Follicle-on-a-chip Engineering. A schematic diagram of the lymphoid follicle-on-a-chip engineering is shown in FIG. 15. It is desired to generate >300 microgels per minute using microfluidic devices. Further, microgel degradation can be obtained within 1-7 days in a user defined manner (FIG. 19). At least 50% survival of immune cells was obtained in the microgels (FIG. 20).

### Lymphoid Tissue-Integrated Gut Organoids

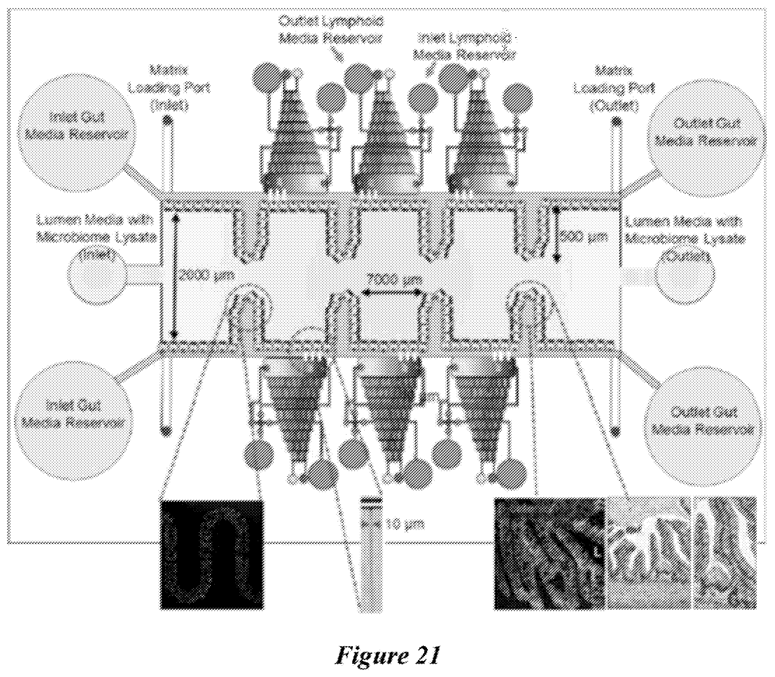

It is desired to develop lymphoid tissue-integrated gut organoids to study vaccine mediated immunity (FIG. 21).

The experimental approach involves generating lymphoid tissue-integrated gut organoids, validating the organoids, and assessing vaccine response (e.g., influenza vaccine response). Tubular mini-guts and integrated lymphoid tissues were established. Designer hydrogels were integrated in a perfusable platform to generate a hybrid microchip system. An immunocompetent immune microenvironment that represents B cell follicles of lymph nodes and first line of defense was engineered. Microbiome was integrated. Organoids were validated using state-of-the-art omics, sequencing, and microscopy.

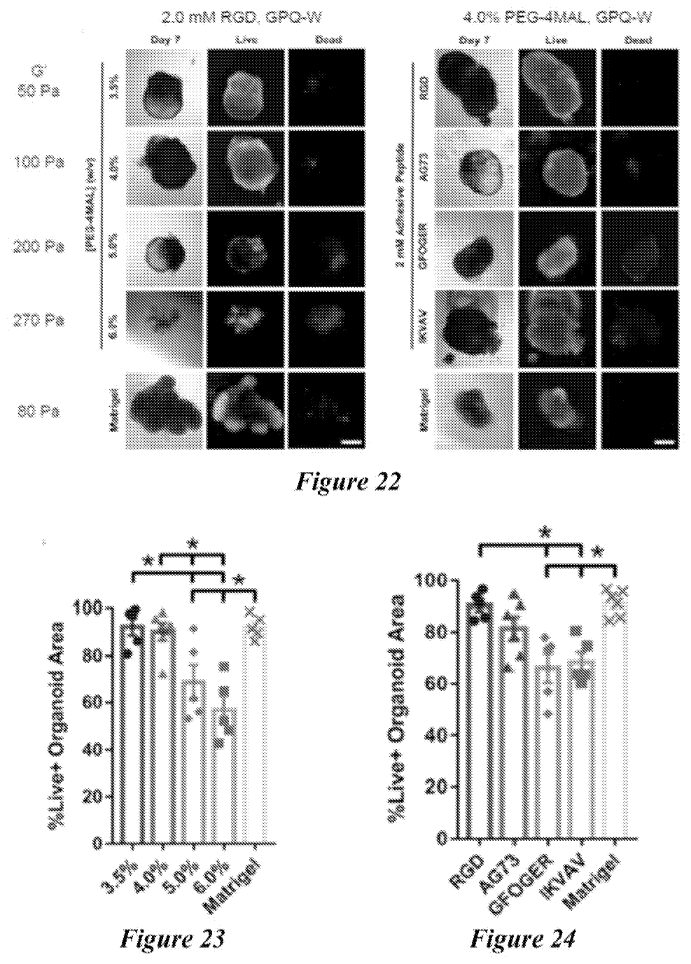

Human Intestinal Organoids. PEG-4MAL density and adhesive peptide control was investigated (FIG. 22). HIO viability and development was validated for hiPSC in PEG-4MAL (FIG. 23 and FIG. 24).

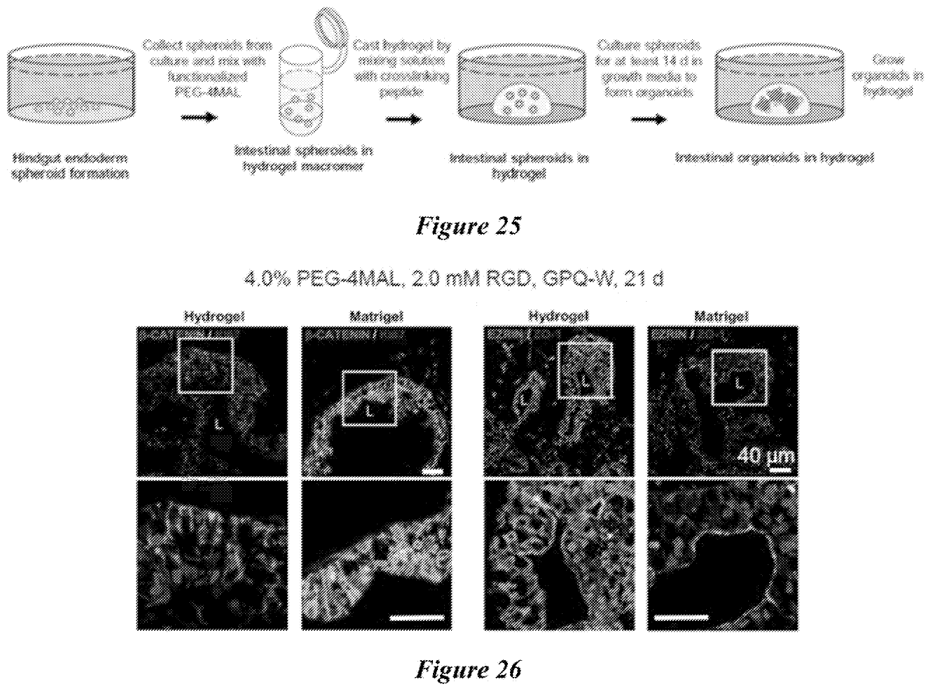

PEG-4MAL density regulates HIO generation. The effect of PEG-4MAL density on HIO generation was investigated and validated with HiPSCs (FIG. 25). Polarized distribution of apical EZRIN and basolateral β-CATENIN was observed as well as expression of ZO-1 and ECAD in the apical junctional complex (FIG. 26).

In moving the technology to gut-on-a-chip, a perfusable microfluidic system is needed, which is difficult to achieve with PEG-4MAL. PEG-4MAL gels quickly, meaning that laser dissection can be needed to form a perfusable microfluidic system, which is a roadblock to easy manufacturing.

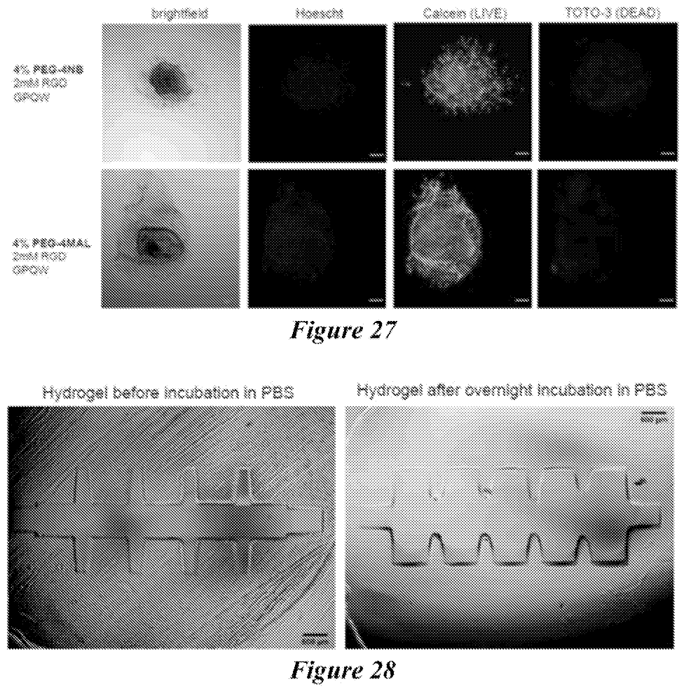

Characterize intestinal cells from hiPSCs grown in hydrogel. Intestinal cells from hiPSCs grown in hydrogel were characterized (FIG. 27). Spheroid-to-HIO (d14) structures exhibited high variability after 24 hours grown in 4% PEG-4NB and 4% PEG-4MAL (FIG. 27). The impact of the presence of immune cells on differentiation is also being investigated.

Fabrication of Gut Organoid-on-a-chip devices. Gut organoid-on-a-chip microchip systems comprise an elastomeric device with a central hydrogel chamber for subsequent organoid culture and perfusion (FIG. 28). Optimization of fabrication parameters to obtain crosslinked gut-hydrogel systems with high shape fidelity and controlled swelling is being investigated.

Optimization of swelling characteristics and incorporation of hiPSC-derived intestinal cells is being investigated. Performance of gut organoids when cultures with immune cells is also being investigated.

### Integrated Organ-On-a-Chip

It is desired to develop an integrated organ-on-a-chip model, for example for lymphoid and gut. In some examples, this organ-on-a-chip model can be extended to other mucosal organ systems, such as lungs.

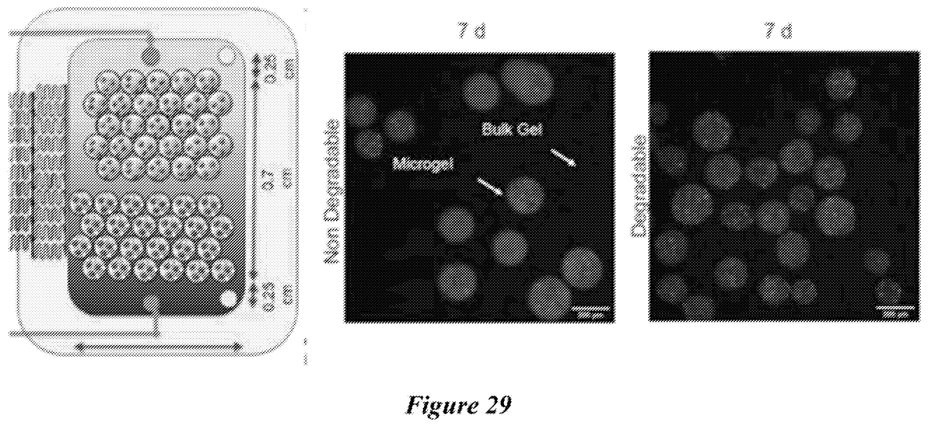

Establishing and characterizing a hybrid bulk and microgel. Composite PEG-4aNB hydrogel bulk and PEG-4MAL microgels containing human B cells were characterized (FIG. 29).

### Example 4—Rapid and Facile Light-Based Approach to Generate Complex Hydrogel Structures for Organ-On-a-Chip Models

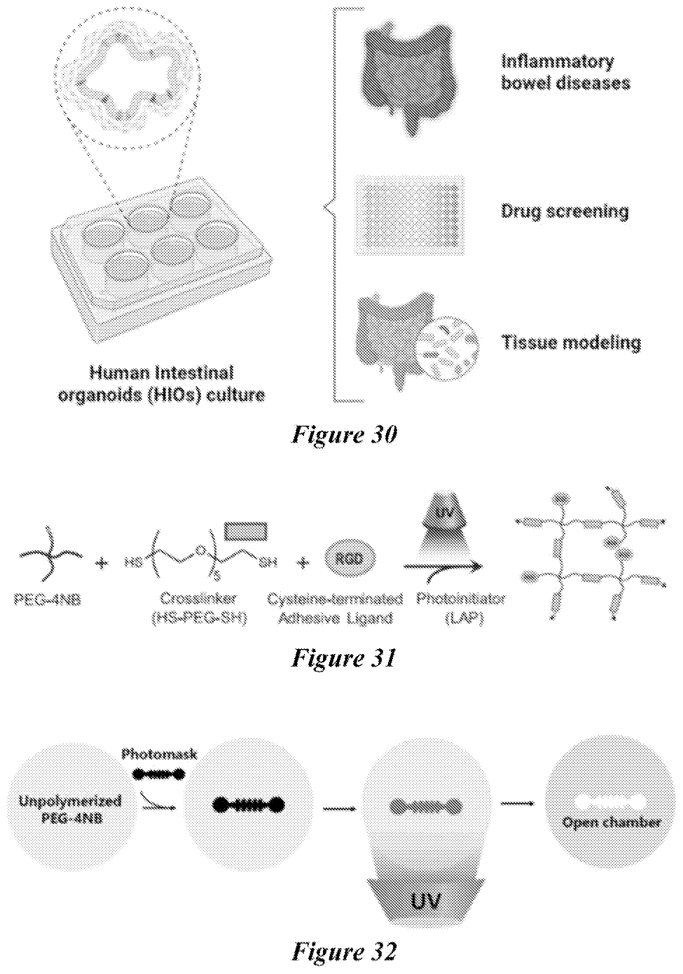

Human intestinal organoids (HIOs) represent an excellent tissue source for intestinal disease and tissue modeling (FIG. 30). HIO derivation in three-dimensional matrices without perfusion can restrict their development and functionality, resulting in closed architectures with reduced growth and homeostasis.

Fabrication of current perfusable gut-on-a-chip platforms based on hydrogels: involve painstaking, time-consuming, and laser-based equipment-intensive methodologies; and are limited to natural, biological matrices (e.g., Matrigel).

Described herein is a rapid and facile light-based approach to generate complex hydrogel structures to use in gut-on-a-chip models. The methods can: reduce preparation times from several hours to seconds; use simple instrumentation; and provide the ability to use synthetic hydrogels over natural matrices, which provides flexibility and lowers regulatory burdens.

Methods. A schematic diagram of the synthetic hydrogel photopolymerization mechanism is shown in FIG. 31. A schematic diagram of photopatterning PEG-4NB hydrogels for the fabrication of perfusable mini-gut structures using UV-light and a photomask is shown in FIG. 32. Photopatterning of complex structures in synthetic hydrogels including perfusable channels for cell culture and media perfusion can be accomplished in less than 1 second.

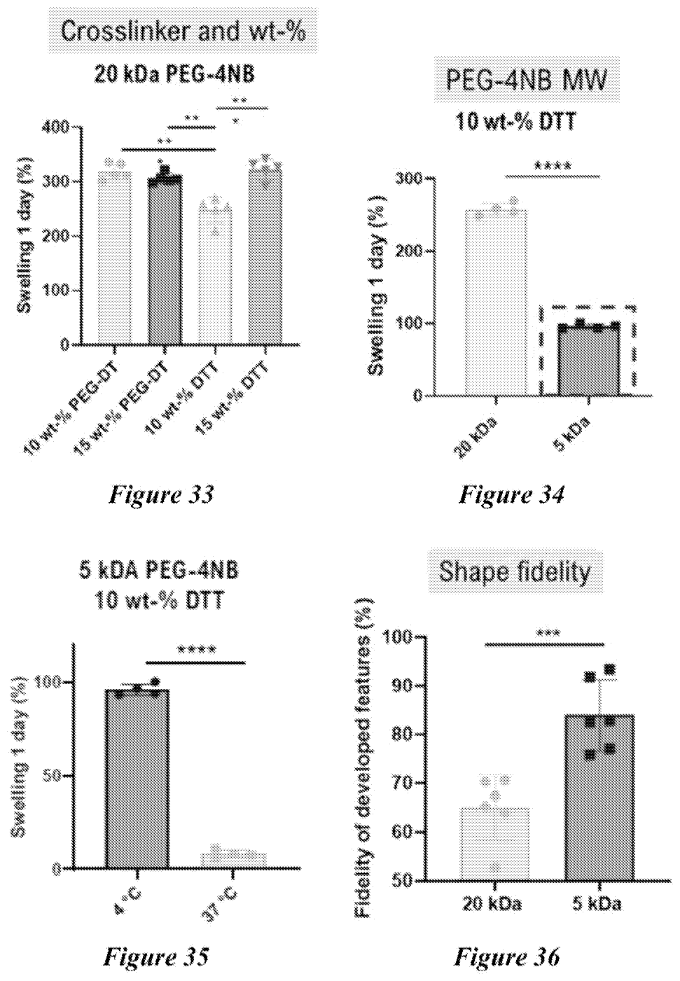

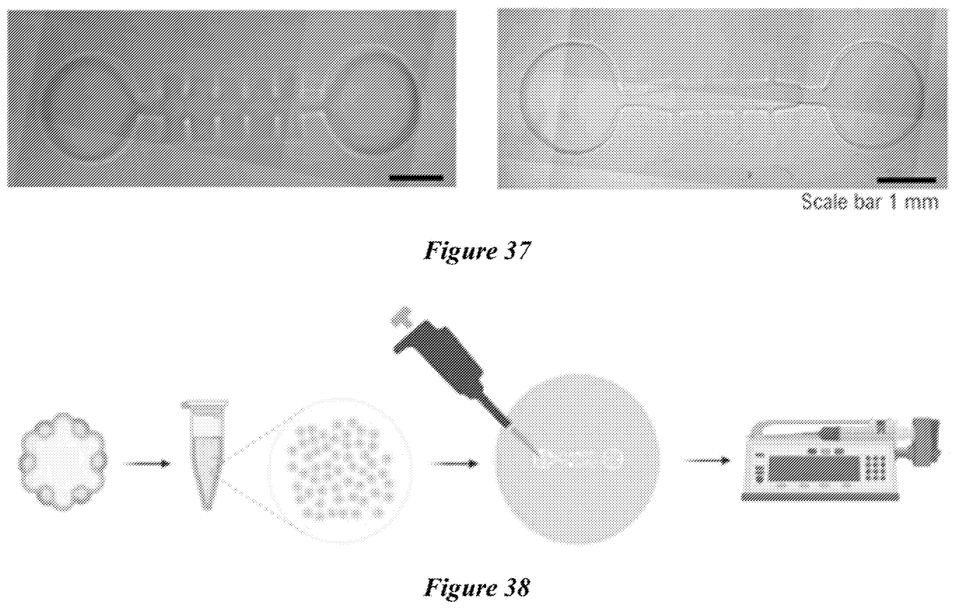

Photopatterning hydrogels with controlled swelling and shape fidelity. Controlling hydrogel swelling can be a challenge. Swelling can be a key parameter to maintain high shape fidelity in the hydrogel features. The effect of crosslinker and wt. % (FIG. 33), PEG-4NB molecular weight (FIG. 34), and temperature (FIG. 35) on swelling were investigated. The hydrogel formulation provides reduced swelling (8±2%), which leads to high shape fidelity (83±8%) of photopatterned features with different geometries (FIG. 36). Different photomask designs can be used to create complex geometries (FIG. 37).

Seeding HIOs in gut-on-a-chip. A schematic diagram of seeding HIOs in gut-on-a-chip is shown in FIG. 38. Single cells of HIOs at day 28 were used for seeding in the devices (5-10×10⁶ cells/mL).

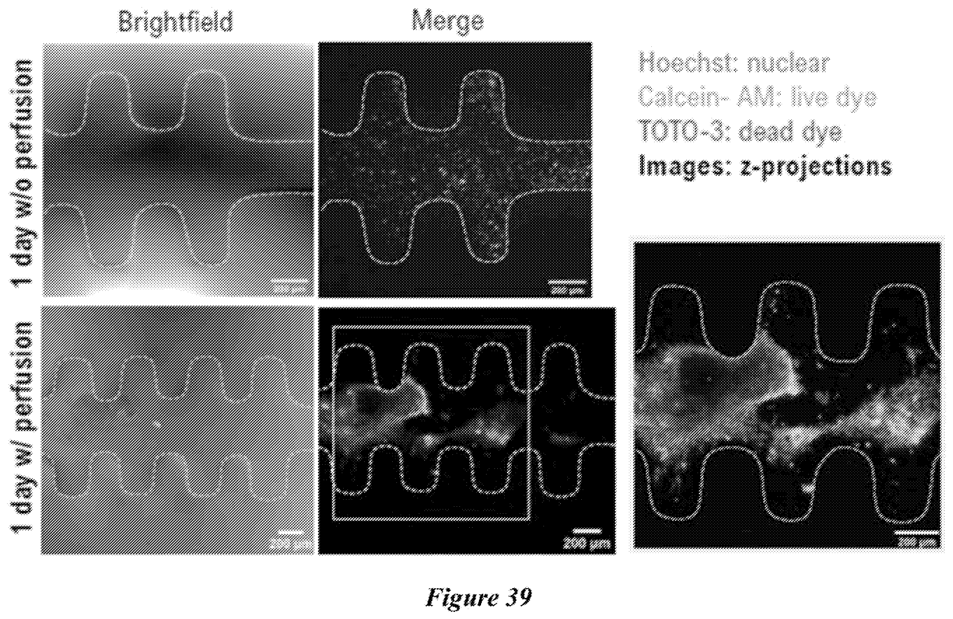

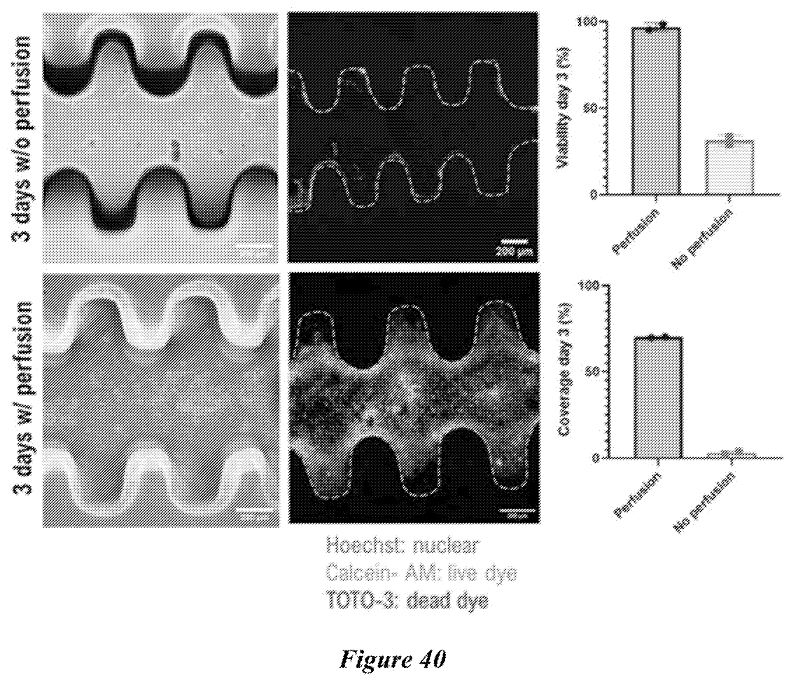

Culturing single cells of HIOs in gut-on-a-chip. Media perfusion can be important for cell survival over time (FIG. 39-FIG. 40). Media perfusion improves cell viability and device % surface coverage over time (FIG. 40).

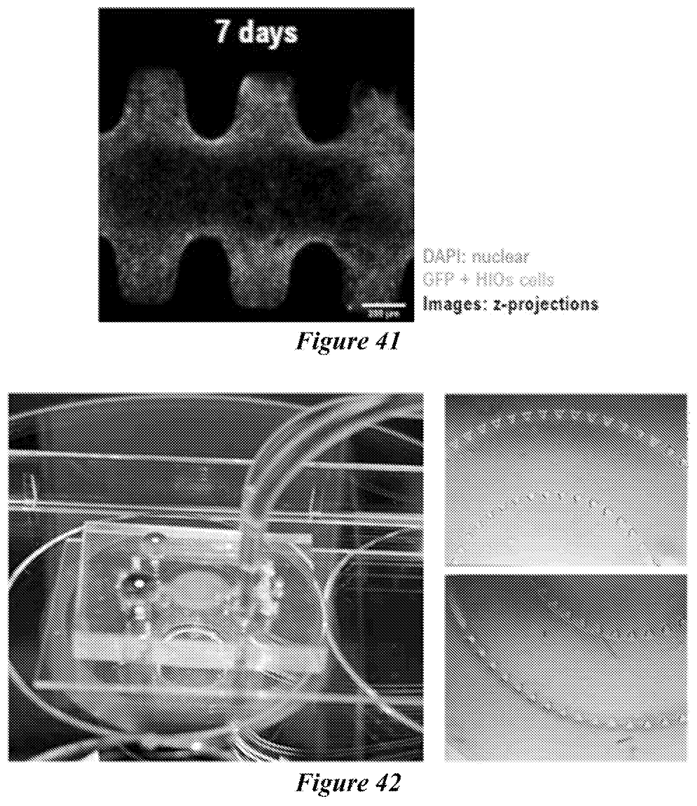

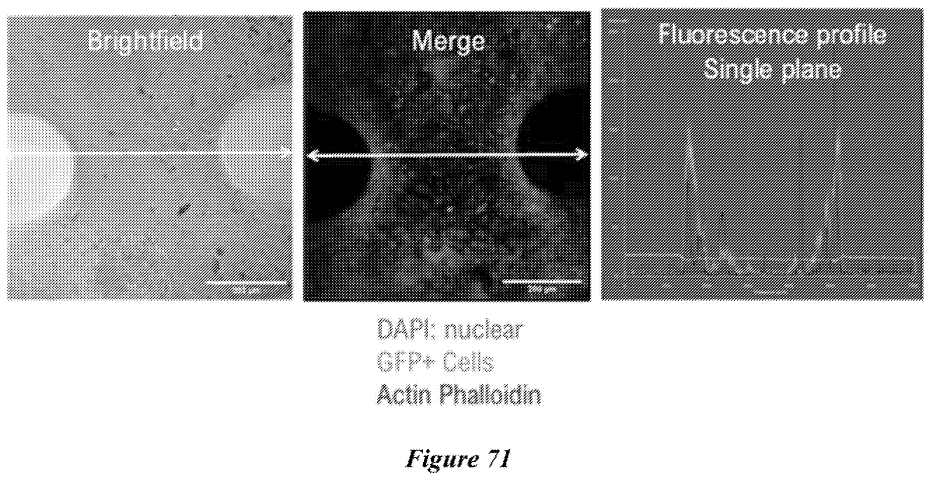

Gut-on-a-chip devices allow long-term culture of HIOs; HIOs grow colonizing hydrogel surfaces (FIG. 41 and FIG. 71).

Conclusion. These results demonstrated that the gut-on-a-chip systems are suitable and accessible platforms for the development of relevant intestinal organoids as well as long-term culture systems. Further experiments can focus on the evaluation of cell type distribution and intestinal functional studies.

### Example 5

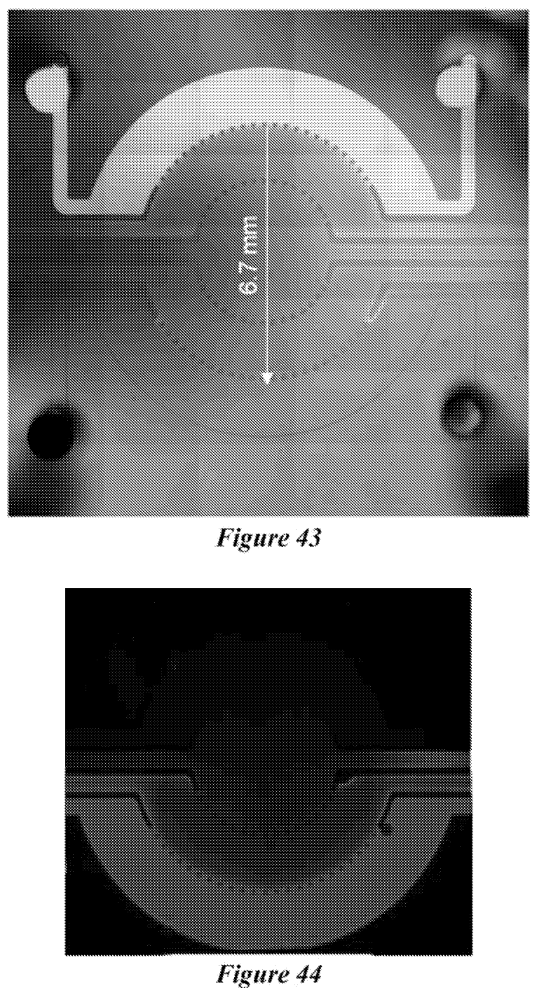

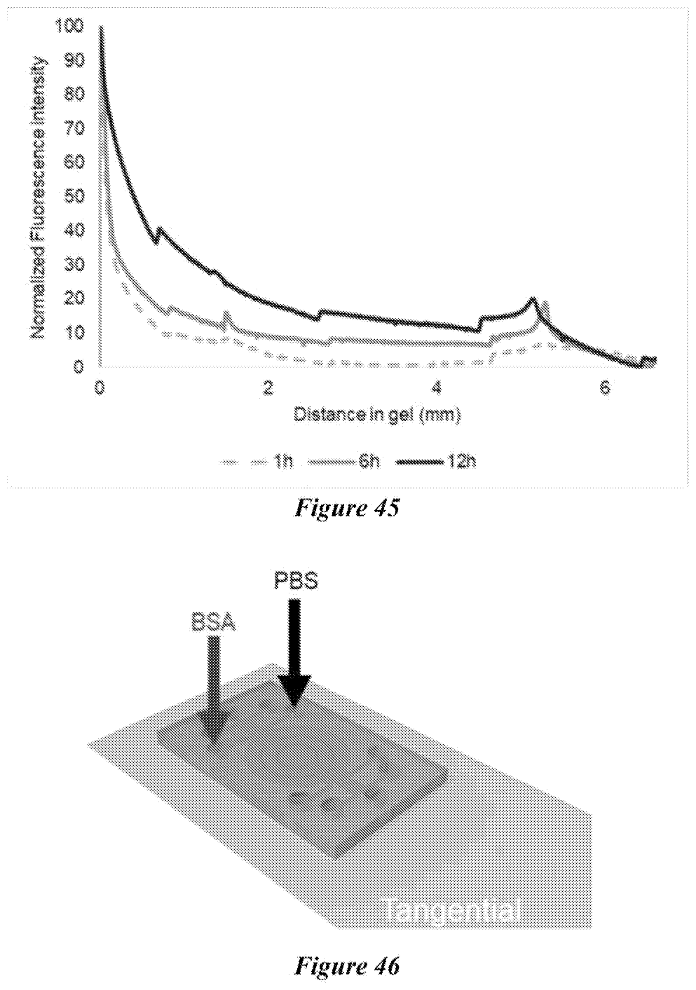

Establish chemokine gradients within polymer PEG-4NB network for cell migration. A bullseye design was created where gel was placed in the 2 innermost rings and flow circulates in the outermost ring (FIG. 42-FIG. 45). The design allows for flow to go around the gel without disrupting it. Flow can go across the device without backflow on inlets. PEG-4NB gel (7.5% w/v, 100% DDT) was used inside the bullseye design.

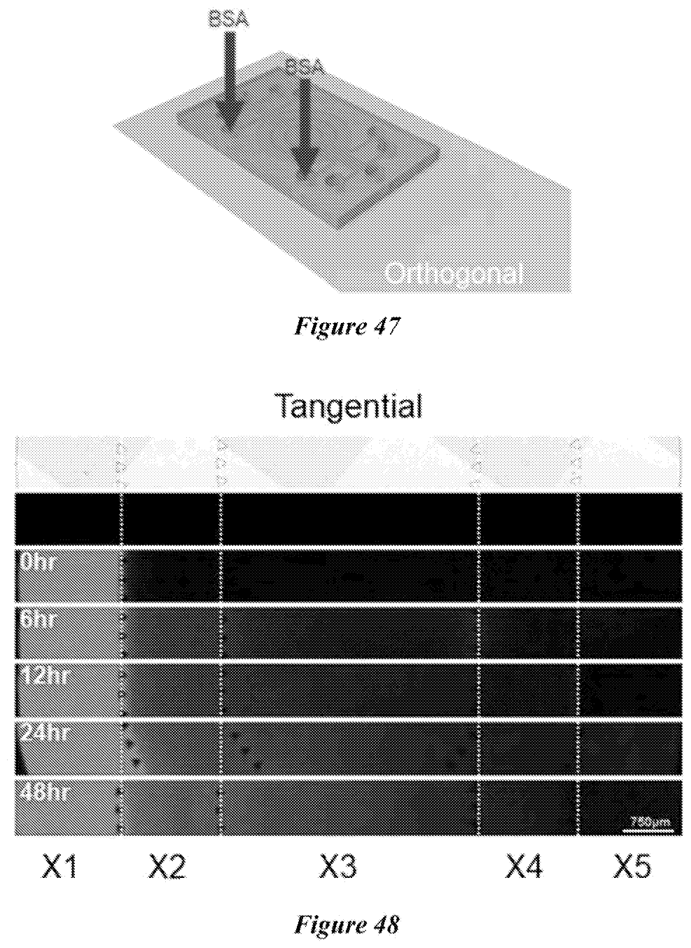

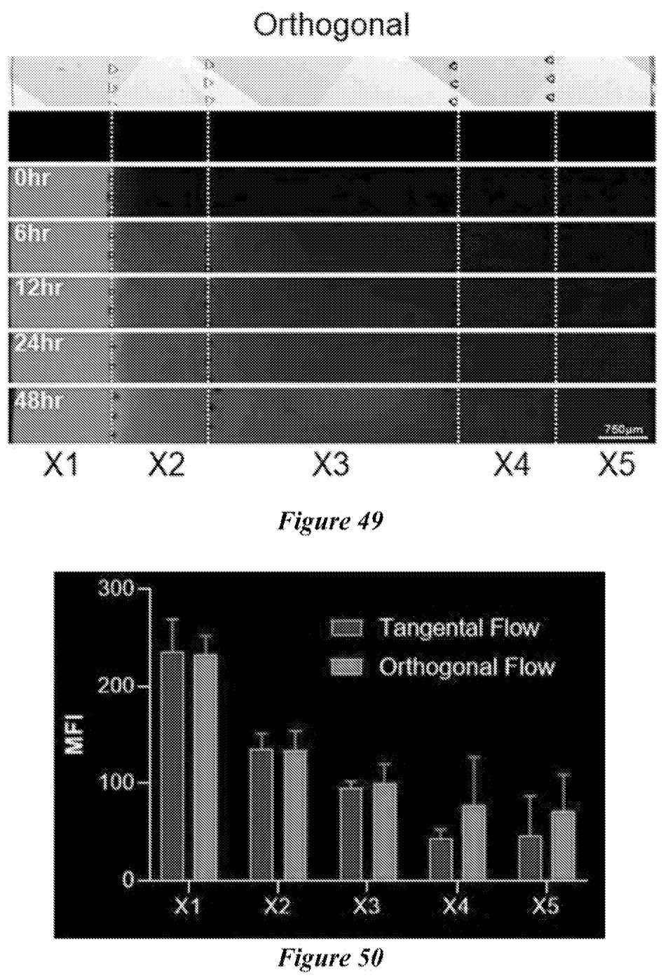

Establish in situ stability of new PEG-4NB in microfluidics for 4 days. Gel was placed in the innermost 2 rings. A comparison was made between utilizing a tangential flow and an orthogonal flow pattern (FIG. 46-FIG. 50). The gel was PEG-4NB (7.5% w/v; VPM:DTT 80:20). A BSA gradient was tagged across the gel over 48 hours. The gradient travels the full diameter of the device. The experiment established the in situ stability of PEG-4NB in microfluidics for 4 days.

B cell maturation in PEG-4NB bulk gels. Experiments were performed with the intent of demonstrating at least 50% survival of B cells, 50% or more cells acquire characteristic phenotypes of GC B cells, and at least 10% of cells acquire memory or plasmablast phenotype in the initial cultures. The Experimental design included: 400 k PBMCs, 40 k CD40LL, and 40 k FDC/HK cells in 40 μL of gel; 50% VPM, 0.3 mM GFOGER and 0.7 mM RDG; media: IL4 (D0-D2), H1N1 antigen (D0-D2), IL21 (D2-D12), and nBAFF (D0-D12). The CD40LL and FDC/HK cells are present as engineered cells or through soluble or bead-based functionalization of proteins, peptides, or other biomolecules.

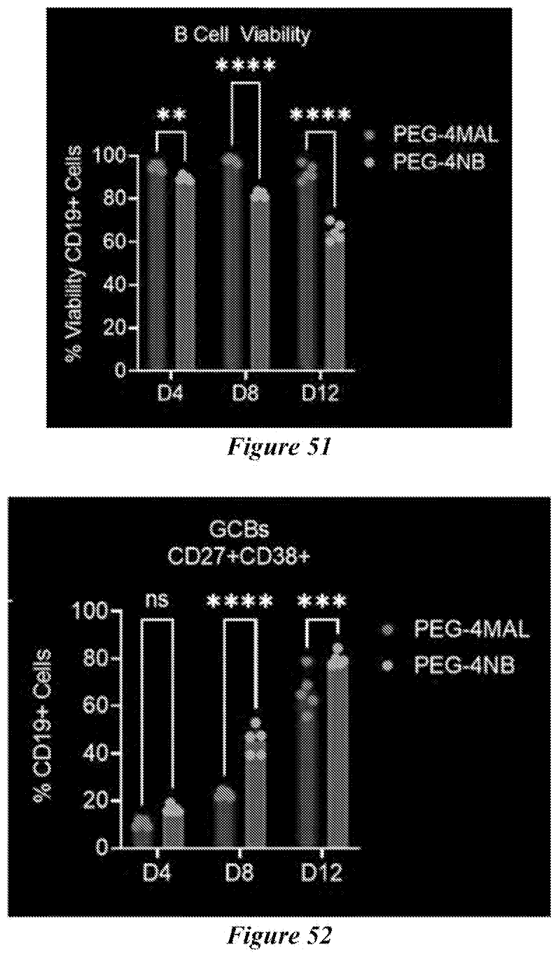

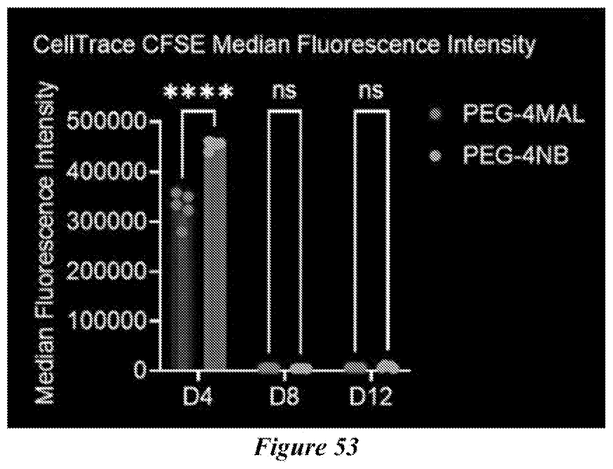

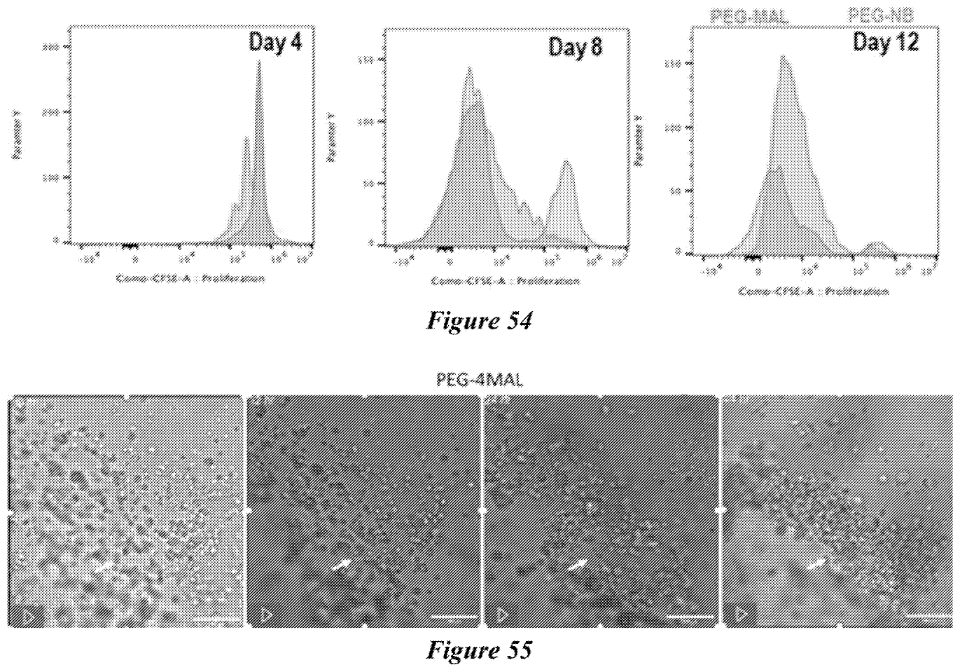

The results indicated that: PEG-4NB supports >50% B cell survival and proliferation; human PBMC B cells can differentiate in PEG-4NB cells (>50% into GC B cells); and PEG-4NB outcomes were within 10-50% of those for PEG-4MAL (FIG. 51-FIG. 54).

Incorporation of Fibrinogen/Collagen in PEG-4NB. The compositions contained PEG-4MAL/4NB (7.5% w/v), REDV (3.0 mM), VPM, and DTT (VPM:DTT=75:25). Compositions further contained fibrinogen (4 mg/ml), collagen (1 mg/ml), and/or thrombin (2 u/ml). Compositions further included PEG-4NB:Fibrin/collagen at ratios of 1:0, 3:1, or 1:1 by volume.

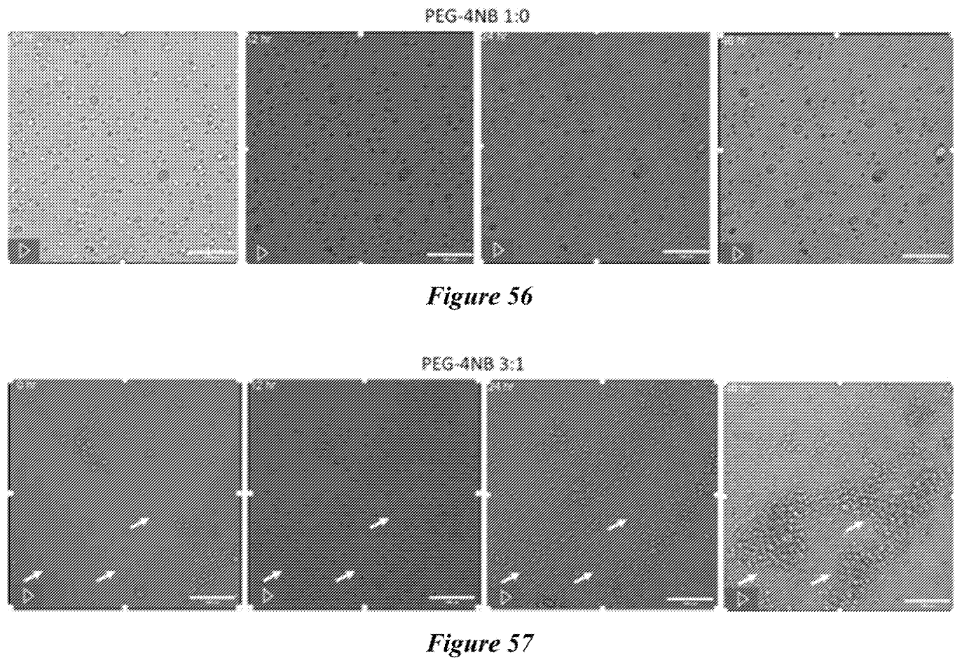

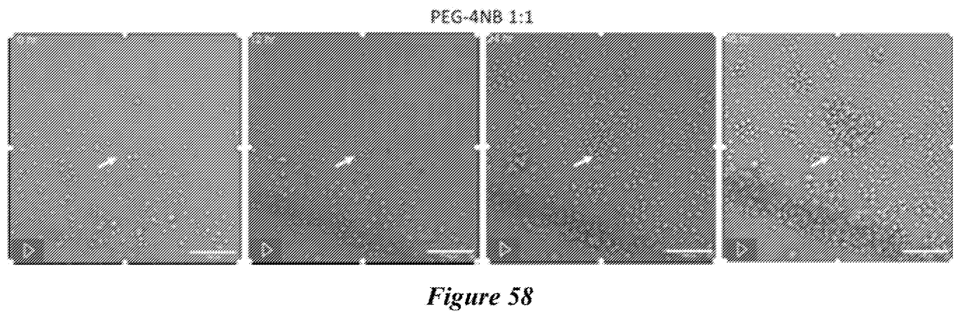

The addition of a secondary polymer network was initially investigated in BUMBLE-B. PEG-4NB with fibrin and collagen remains injectable and supports cell proliferation and clustering; this was demonstrated through B cell model cell line LY3 clustering in the presence of CD40LL (FIG. 55-FIG. 58).

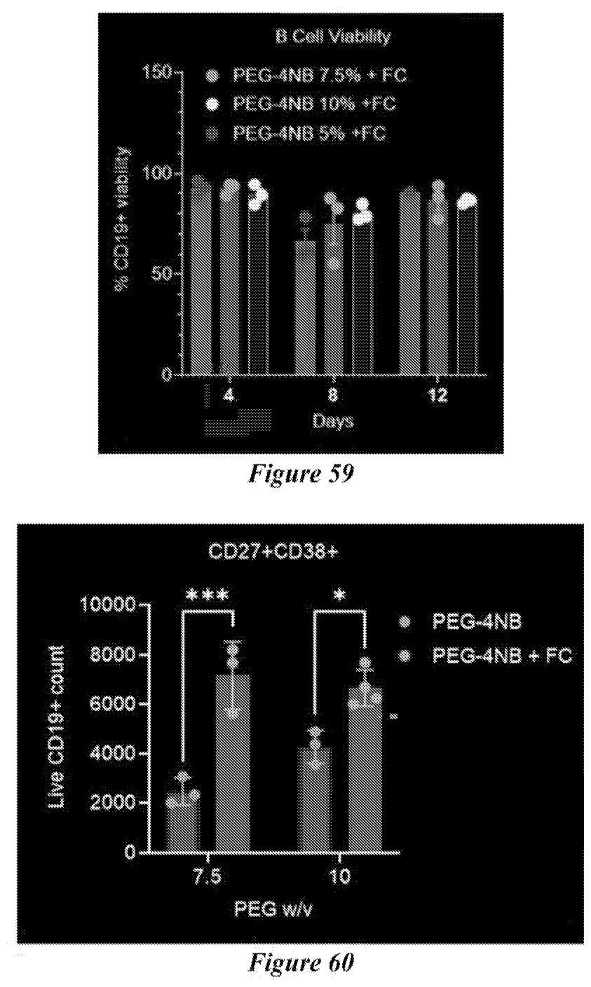

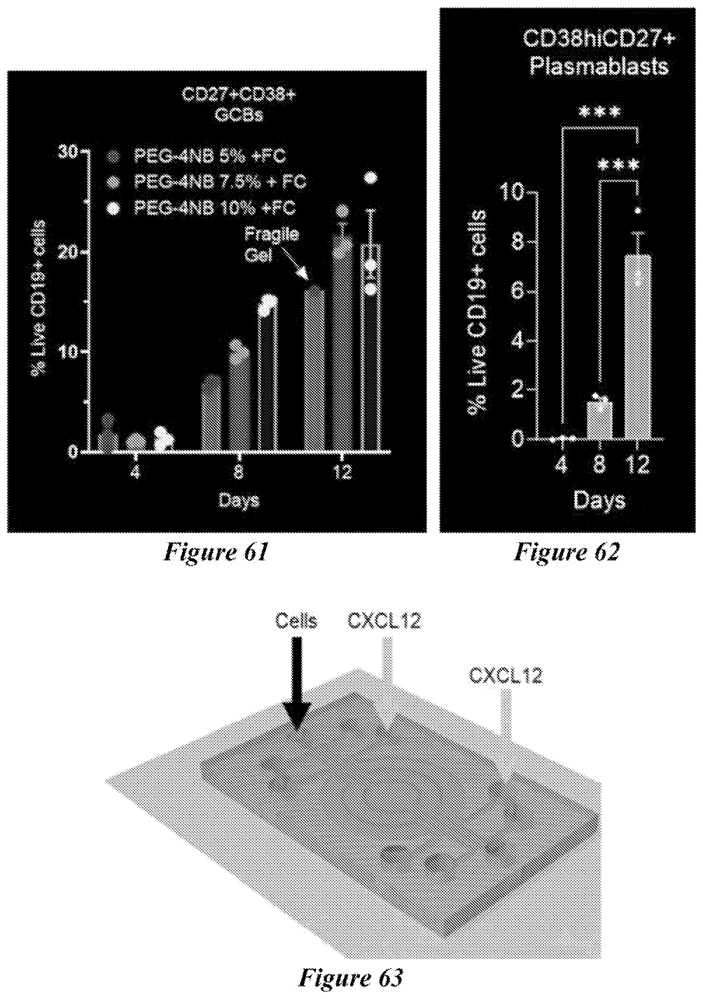

B cell maturation in PEG-4NB+FC gels in device. The count of live B cells increased in PEG-4NB hydrogels with fibrinogen and collagen (FC)(FIG. 59-FIG. 62). Based on these results, the use of PEG-4NB+FC will be used in experiments in lymphoid follicle on chip. The fibrinogen and collagen are examples, however other devices comprising synthetic gel-ECM protein blends can be used, for example with other synthetic or natural polymers used in place of fibrinogen and/or collagen.

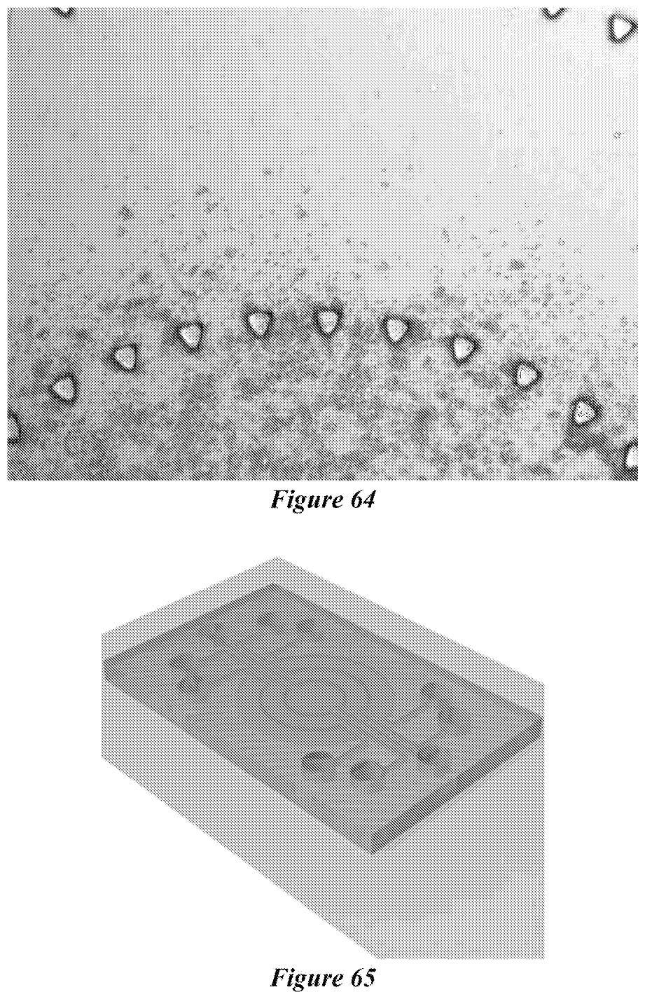

Demonstration of cell migration within device. Device comprise PEG-4NB (5% w/v), REDV (3.0 mM), VPM, and DTT (VPM:DTT=80:20). Compositions further contained fibrinogen (4 mg/ml), collagen (1 mg/ml), and/or thrombin (2 u/ml). A schematic diagram of the device is shown in FIG. 63. Human B and T cell migration induced by CXCL12 gradient with 4 days (FIG. 64).

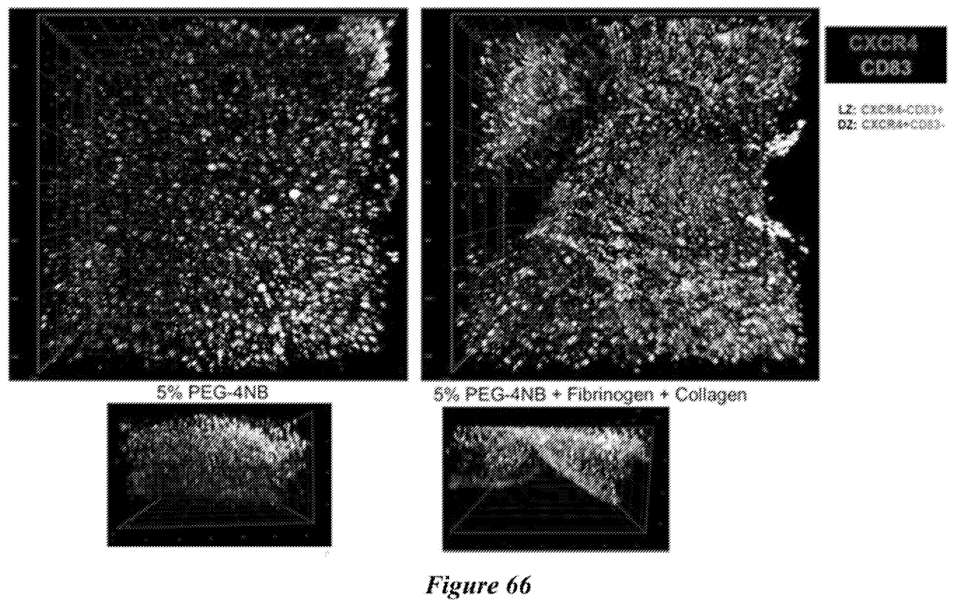

Immune cell differentiation. Cell differentiation was compared in a device (FIG. 65) comprising PEG-4NB relative to one comprising PEG-4NB, fibrinogen, and collagen. The results indicated that immune cell differentiation was achieved as distinct phenotype compartments in the device comprising PEG-4NB, fibrinogen, and collagen (FIG. 66).

### Example 6—Rapid and Facile Light-Based Approach to Generate Complex Hydrogel Structures for Organ-On-a-Chip Model

Inflammatory bowel disease (IBD) is a term for two conditions (Crohn's disease and ulcerative colitis) that are characterized by chronic inflammation of the gastrointestinal tract. IBD affects more than 6 million people worldwide. The exact cause of IBD is unknown.

Human intestinal organoids (HIOs) represent an excellent tissue source for IBD and tissue modeling (FIG. 30).

Traditional human intestinal organoid derivation takes place in Matrigel (trade name for the solubilized basement membrane matrix secreted by Engelbreth-Holm-Swarm (EHS) mouse sarcoma cells produced by Corning Life Sciences) and static conditions. This can result in a restricted development and functionalities of the final organoids, such as resulting in closed architectures with reduced growth and homeostasis. This indicates the necessity of perfusable systems to obtain more functional and relevant intestinal models.

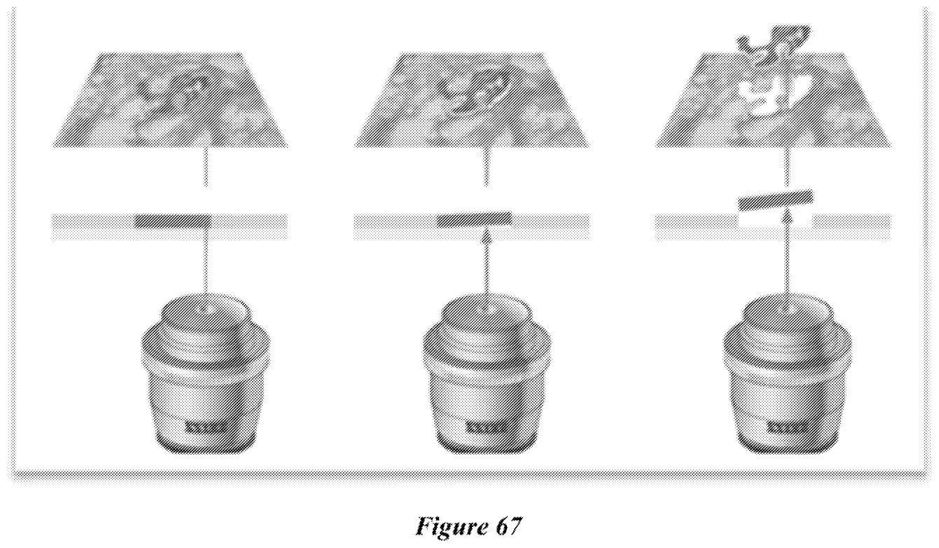

Currently, the fabrication of perfusable gut on a chip platforms using hydrogels involve painstaking, time-consuming, and laser-based equipment-intensive methodologies and are limited to natural, biological matrices (e.g., Matrigel). For example, the fabrication of current perfusable gut on a chip platforms using hydrogels are based on the patterning of natural-based matrices using PALM microbeam Zeiss microscope as a dissection tool (FIG. 67) (Nikolaev M, Mitrofanova et al. Homeostatic mini-intestines through scaffold-guided organoid morphogenesis. Nature. 2020 September; 585(7826):574-578).

Described herein are methods for the photopatterning of PEG-4NB hydrogels using UV light and a photomask (FIG. 32). Particularly, an unpolymerized polymer solution is covered with a photomask with the desired design and then irradiated with UV light. The section of the polymer that is covered with the features of the photomask will remain uncrosslinked, while the polymer that is exposed to the UV light will be crosslinked. The uncrosslinked polymer can be washed away to obtain complex structures patterned in the hydrogel, including perfusable channels for cell culture and media perfusion (FIG. 37). This technique reduces preparation times from hours to seconds and uses very simple instrumentation. In addition, the possibility of using synthetic polymers in these methods provides flexibility and lowers regulatory burdens relative to methods using natural matrices.

One of the big challenges was to control the swelling of the hydrogels to maintain a high shape fidelity of the developed features. For this, different polymer formulations were analyzed. The influence of the crosslinking agent, polymer concentration and molecular weight, and temperature were studied (FIG. 33-FIG. 36). A polymer formulation that decreased the swelling and provided an excellent shape fidelity was obtained: PEG-4NB 5 kDa at 10 wt. % crosslinked with DTT. This hydrogel formulation provide a reduced swelling, which leads to high shape fidelity (83±8%) of photopatterned features with different geometries.

The next step was to culture single cells obtained from HIOs in the “gut-on-a chip” devices. Single cells of HIOs at day 28 were used for seeding in the devices (5-10×10⁶ cells/mL). It was observed that after 3 days of culture, the devices that were maintained under constant perfusion showed a larger surface coverage by the cells seeded in the lumen with an excellent viability, in comparison to those that were cultured at static conditions (FIG. 40). In short, media perfusion improves cell viability and device surface coverage over time.

At least 80% device coverage with viable hiPSC-intestinal cells displaying appropriate apical and basolateral marker localization, the presence of differentiated epithelial cells (Paneth, goblet, and enterocytes), fatty acid adsorption, and barrier function with at least 90% FITC-dextran exclusion was demonstrated.

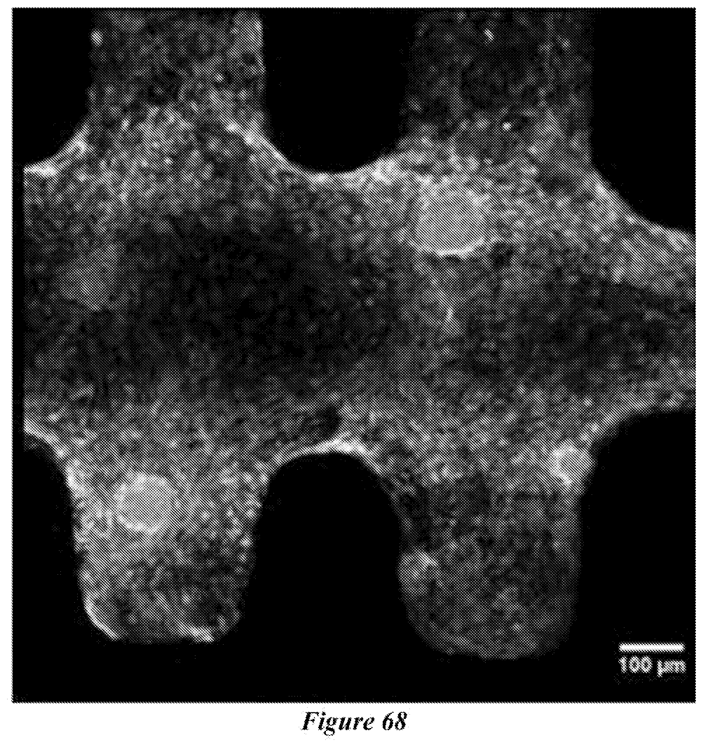

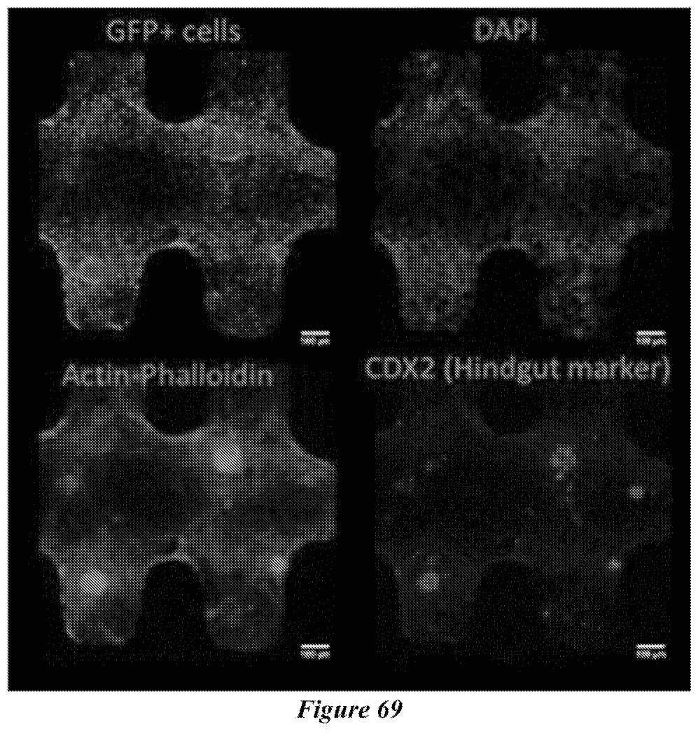

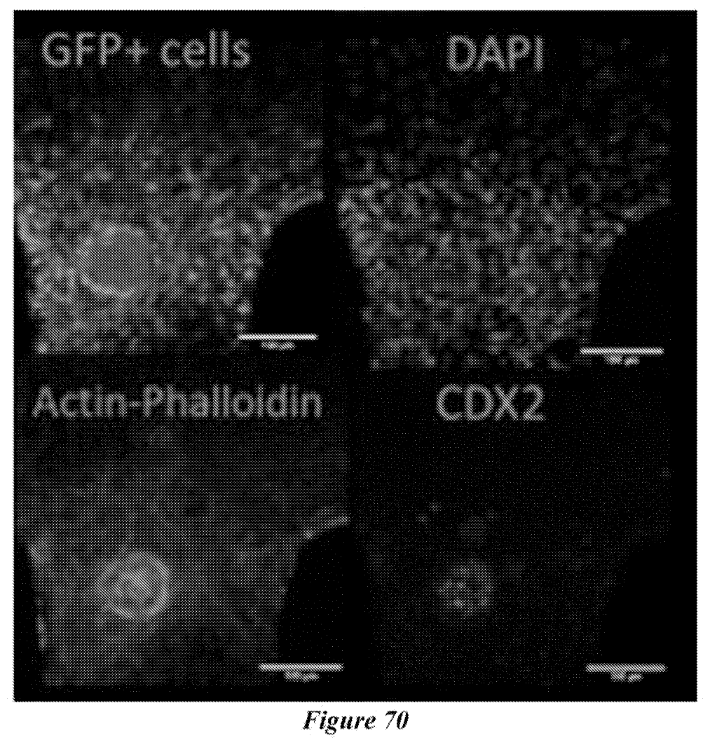

Immunostaining gut on a chip after 4 days in culture is shown in FIG. 68-FIG. 70. After 4 days of culture, some CDX2 positive cells, which is a hindgut marker, were also observed in the cells grown in the devices.

These devices also allowed long-term culture of the cells/HIOs up to 7 days, when the cells start to colonize the device tri-dimensionally, showing a total coverage of the available surface in the device lumen (FIG. 41 and FIG. 71-FIG. 72).

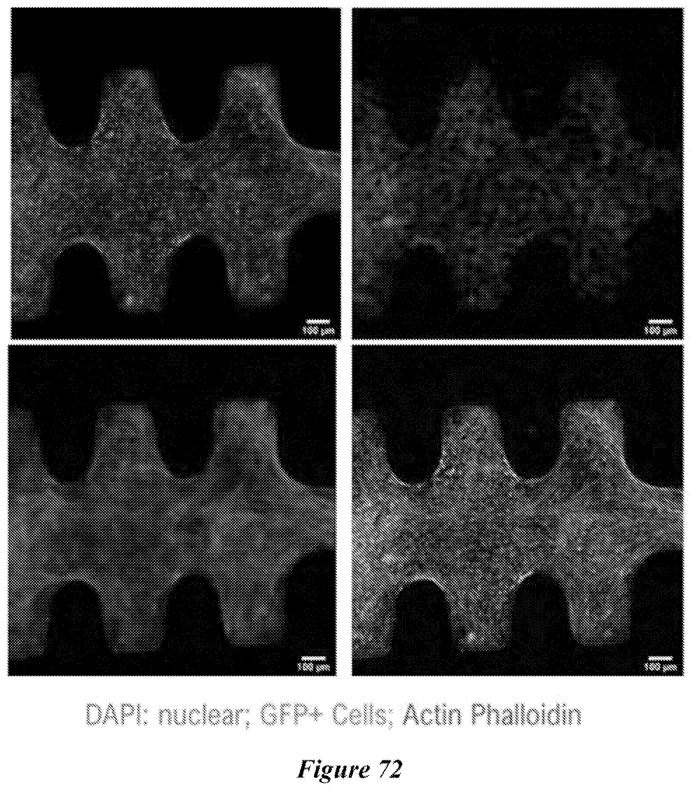

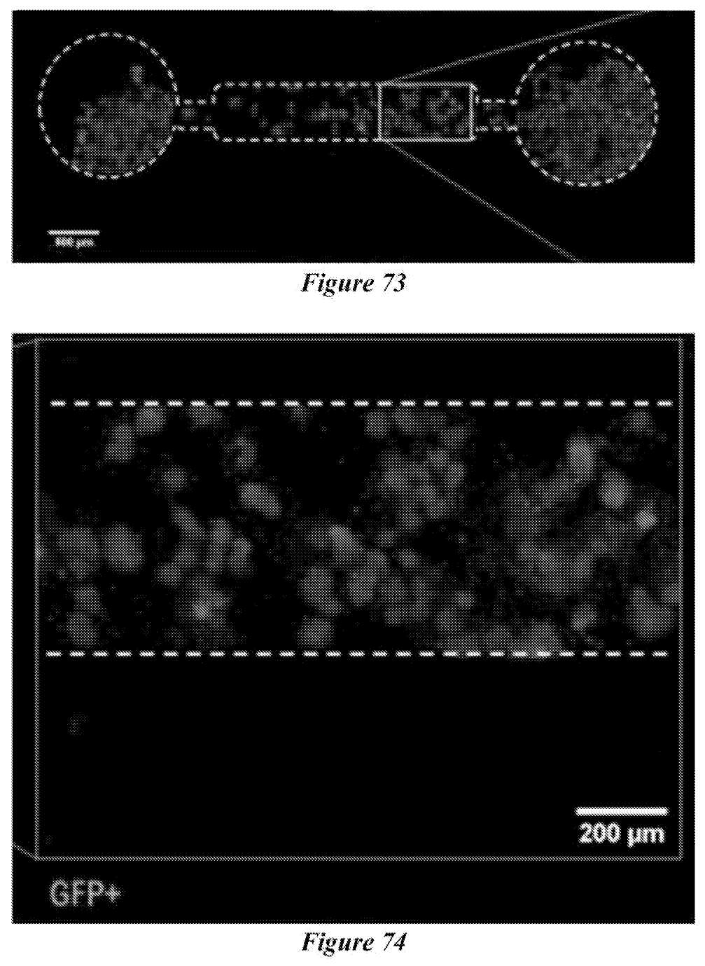

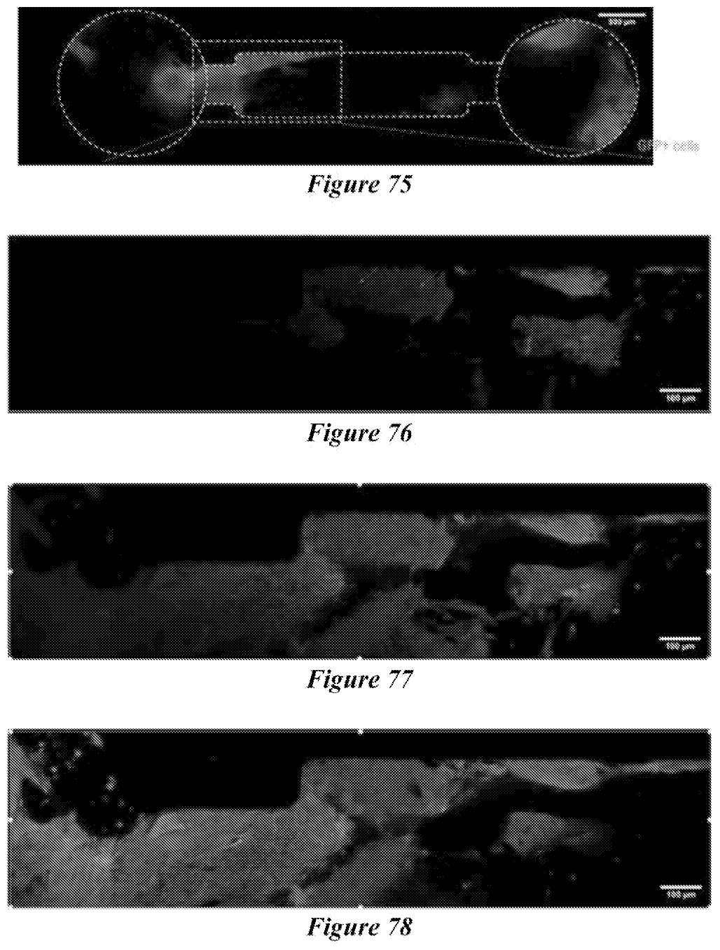

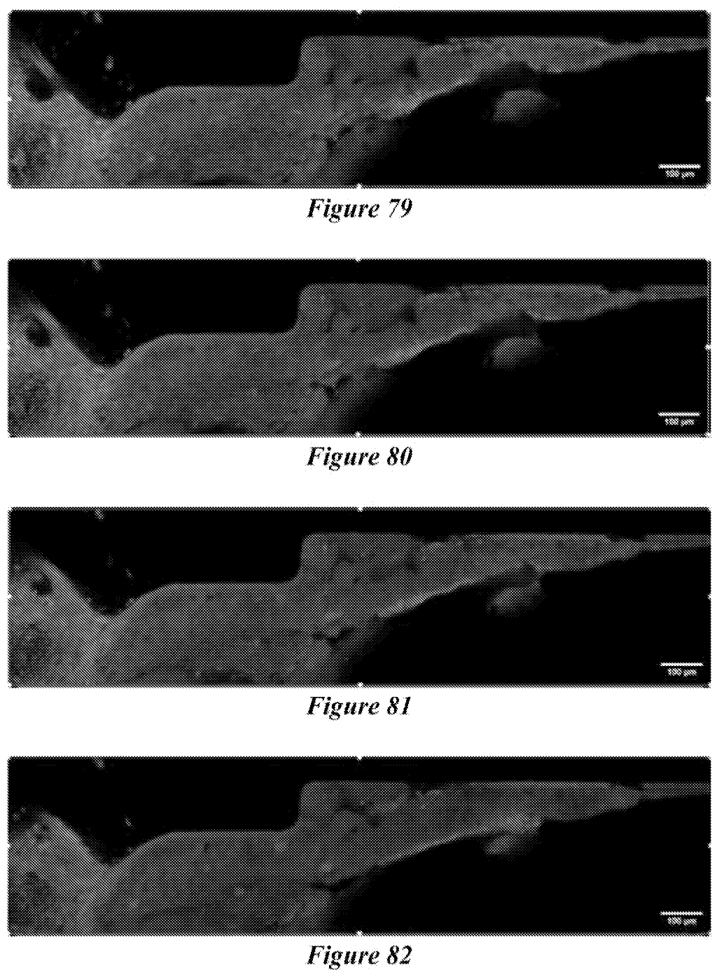

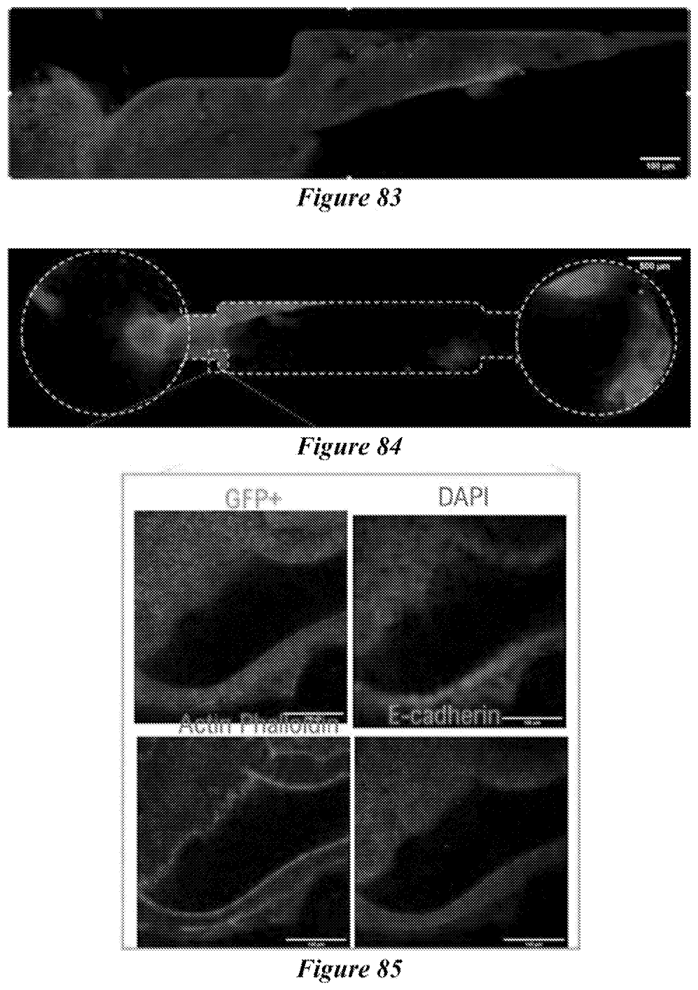

Another interesting approach is to seed intestinal spheroids instead of single cells from HIOs in the devices. Seeding hiPSCs-derived spheroids in tubular gut on a chip device after 24 hours of culture is shown in FIG. 73-FIG. 74. In this case, the formation of 3D complex structures were observed after 6 days of culture (FIG. 75). Initial attachment, coverage, and 3D complex structure formation was observed after 6 days of culture (red arrows, FIG. 76-FIG. 83).

Immunostaining of hiPSCs-derived spheroids in tubular gut on a chip device after 7 days of culture results are shown in FIG. 84-FIG. 85. Positive E-cadherin and CDX2 marker stain suggests the presence of organized epithelial cell regions.

The results indicate that the methods described herein is a powerful tool for accessible, facile, and rapid hydrogel patterning. Gut-on-a-chip systems are suitable platforms for the development of relevant intestinal organoids as well as long-term culture systems.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

The compositions, devices, and methods of the appended claims are not limited in scope by the specific compositions, devices, and methods described herein, which are intended as illustrations of a few aspects of the claims and any methods that are functionally equivalent are intended to fall within the scope of the claims. Various modifications of the compositions, devices, and methods in addition to those shown and described herein are intended to fall within the scope of the appended claims. Further, while only certain representative composition elements, system elements, and method steps disclosed herein are specifically described, other combinations of the composition elements, device elements, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

1. A method of making a device comprising a hydrogel matrix and a first chamber in the hydrogel matrix, the hydrogel matrix being derived from a prepolymer and the first chamber being perfusable, the method comprising:
blocking a first portion of a pre-polymerization solution with a first photomask, the pre-polymerization solution comprising the prepolymer, such that the pre-polymerization solution comprises an exposed portion and a first blocked portion; and
irradiating the exposed portion of the pre-polymerization solution and the first photomask with electromagnetic radiation, the first photomask being substantially opaque to the electromagnetic radiation;
wherein the prepolymer within the exposed portion of pre-polymerization solution photopolymerizes to form the hydrogel matrix and the prepolymer within the first blocked portion does not photopolymerize and forms the first chamber.

2. The method of claim 1, wherein the device further comprises
a second chamber in the hydrogel matrix, the second chamber being perfusable and fluidly independent from the first chamber; and
the method further comprises:
blocking a second portion of the pre-polymerization solution with a second photomask, such that the pre-polymerization solution comprises h exposed portion, the first blocked portion, and a second blocked portion; and
irradiating the exposed portion of the pre-polymerization solution, the first photomask, and the second photomask with electromagnetic radiation, the first photomask and the second photomask being substantially opaque to the electromagnetic radiation;
wherein the prepolymer within the exposed portion of pre-polymerization solution photopolymerizes to form the hydrogel matrix, the prepolymer within the first blocked portion does not photopolymerize and forms the first chamber, and the prepolymer within the second blocked portion does not photopolymerize and forms the second chamber.

3. The method of claim 1, further comprising, after irradiation, removing the first photomask.

4. The method of claim 1, further comprising, after irradiation, rinsing the hydrogel device to remove any remaining pre-polymerization solution and/or prepolymer.

5. The method of claim 1, further comprising disposing the pre-polymerization solution in a mold defining a shape before blocking the first portion of the pre-polymerization solution with the first photomask.

6. The method of claim 1, wherein the hydrogel matrix comprises a synthetic hydrogel.

7-17. (canceled)

18. The method of claim 1, wherein the pre-polymerization solution further comprises a crosslinker and the hydrogel is further derived from the crosslinker.

19. (canceled)

20. (canceled)

21. (canceled)

22. The method of claim 1, wherein the hydrogel matrix is further derived from one or more additional components.

23. The method of claim 22, wherein said one or more additional components comprise one or more additional: monomers, prepolymers, ligands, chemical agents, therapeutic agents, photoinitiators, or a combination thereof.

24. The method of claim 22, wherein the hydrogel matrix is further derived from a natural or biological prepolymer.

25. The method of claim 24, wherein the natural or biological prepolymer comprises fibrinogen, collagen, or a combination thereof.

26. The method of claim 22, wherein the pre-polymerization solution further comprises said one or more additional components.

27. The method of claim 1, wherein the hydrogel matrix further comprises a chemical agent, a therapeutic agent, or a combination thereof dispersed therein, and wherein the chemical agent and/or therapeutic agent has a concentration that varies across the hydrogel matrix, such that the chemical agent and/or the therapeutic agent has a compositional gradient across the hydrogel matrix.

28. (canceled)

29. (canceled)

30. (canceled)

31. The method of claim 1, wherein the electromagnetic radiation comprises UV radiation.

32-35. (canceled)

36. The method of claim 1, wherein the exposed portion of the pre-polymerization solution photopolymerizes in an amount of time of from 1 millisecond to 1 hour.

37. (canceled)

38. The method of claim 1, wherein the hydrogel matrix exhibits a swelling of 10% or less; wherein the hydrogel matrix exhibits a shape fidelity of 50% or more; wherein the hydrogel matrix has a storage modulus of from greater than 0 Pa to 5000 Pa; or a combination thereof.

39-50. (canceled)

51. A device made by the method of claim 1, wherein the device is a microfluidic device.

52. (canceled)

53. A method of use of the device of claim 51 for diagnostics, disease modeling, regenerative medicine, drug screening, tissue modeling, or a combination thereof.

54. A method of use of the device of claim 51 as a biomaterial substrate or scaffold, a cell culture substrate or platform, or a combination thereof.

55-58. (canceled)

59. The method of claim 54, wherein the method comprises using the device to grow an organoid and/or three-dimensional cell clusters.

60-68. (canceled)
